SEQUENCE LISTING (9 sequences)

SEQ ID NO: 1 - protein, 351 aa, Tequatrovirus T4
MKIAIINMGNNVINFKTVPSSETIYLFKVISEMGLNVDIISLKNGVYTKSFDEVDVNDYDRLIVVNSSINFFGGKPNLAILSAQKFMAKYKSKIYYLFTDIRLPFSQSWPNVKNRPWAYLYTEEELLIKSPIKVISQGINLDIAKAAHKKVDNVIEFEYFPIEQYKIHMNDFQLSKPTKKTLDVIYGGSFRSGQRESKMVEFLFDTGLNIEFFGNAREKQFKNPKYPWTKAPVFTGKIPMNMVSEKNSQAIAALIIGDKNYNDNFITLRVWETMASDAVMLIDEEFDTKHRIINDARFYVNNRAELIDRVNELKHSDVLRKEMLSIQHDILNKTRAKKAEWQDAFKKAIDL

SEQ ID NO: 2 - protein, 400 aa, Tequatrovirus T4
MRICIFMARGLEGCGVTKFSLEQRDWFIKNGHEVTLVYAKDKSFTRTSSHDHKSFSIPVILAKEYDKALKLVNDCDILIINSVPATSVQEATINNYKKLLDNIKPSIRVVVYQHDHSVLSLRRNLGLEETVRRADVIFSHSDNGDFNKVLMKEWYPETVSLFDDIEEAPTVYNFQPPMDIVKVRSTYWKDVSEINMNINRWIGRTTTWKGFYQMFDFHEKFLKPAGKSTVMEGLERSPAFIAIKEKGIPYEYYGNREIDKMNLAPNQPAQILDCYINSEMLERMSKSGFGYQLSKLNQKYLQRSLEYTHLELGACGTIPVFWKSTGENLKFRVDNTPLTSHDSGIIWFDENDMESTFERIKELSSDRALYDREREKAYEFLYQHQDSSFCFKEQFDIITK

SEQ ID NO: 3 - protein, 1616 aa, Homo sapiens
MPARTAPARVPTLAVPAISLPDDVRRRLKDLERDSLTEKECVKEKLNLLHEFLQTEIKNQLCDLETKLRKEELSEEGYLAKVKSLLNKDLSLENGAHAYNREVNGRLENGNQARSEARRVGMADANSPPKPLSKPRTPRRSKSDGEAKPEPSPSPRITRKSTRQTTITSHFAKGPAKRKPQEESERAKSDESIKEEDKDQDEKRRRVTSRERVARPLPAEEPERAKSGTRTEKEEERDEKEEKRLRSQTKEPTPKQKLKEEPDREARAGVQADEDEDGDEKDEKKHRSQPKDLAAKRRPEEKEPEKVNPQISDEKDEDEKEEKRRKTTPKEPTEKKMARAKTVMNSKTHPPKCIQCGQYLDDPDLKYGQHPPDAVDEPQMLTNEKLSIFDANESGFESYEALPQHKLTCFSVYCKHGHLCPIDTGLIEKNIELFFSGSAKPIYDDDPSLEGGVNGKNLGPINEWWITGFDGGEKALIGFSTSFAEYILMDPSPEYAPIFGLMQEKIYISKIVVEFLQSNSDSTYEDLINKIETTVPPSGLNLNRFTEDSLLRHAQFVVEQVESYDEAGDSDEQPIFLTPCMRDLIKLAGVTLGQRRAQARRQTIRHSTREKDRGPTKATTTKLVYQIFDTFFAEQIEKDDREDKENAFKRRRCGVCEVCQQPECGKCKACKDMVKFGGSGRSKQACQERRCPNMAMKEADDDEEVDDNIPEMPSPKKMHQGKKKKQNKNRISWVGEAVKTDGKKSYYKKVCIDAETLEVGDCVSVIPDDSSKPLYLARVTALWEDSSNGQMFHAHWFCAGTDTVLGATSDPLELFLVDECEDMQLSYIHSKVKVIYKAPSENWAMEGGMDPESLLEGDDGKTYFYQLWYDQDYARFESPPKTQPTEDNKFKFCVSCARLAEMRQKEIPRVLEQLEDLDSRVLYYSATKNGILYRVGDGVYLPPEAFTFNIKLSSPVKRPRKEPVDEDLYPEHYRKYSDYIKGSNLDAPEPYRIGRIKEIFCPKKSNGRPNETDIKIRVNKFYRPENTHKSTPASYHADINLLYWSDEEAVVDFKAVQGRCTVEYGEDLPECVQVYSMGGPNRFYFLEAYNAKSKSFEDPPNHARSPGNKGKGKGKGKGKPKSQACEPSEPEIEIKLPKLRTLDVFSGCGGLSEGFHQAGISDTLWAIEMWDPAAQAFRLNNPGSTVFTEDCNILLKLVMAGETTNSRGQRLPQKGDVEMLCGGPPCQGFSGMNRFNSRTYSKFKNSLVVSFLSYCDYYRPRFFLLENVRNFVSFKRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEVRNGASALEISYNGEPQSWFQRQLRGAQYQPILRDHICKDMSALVAARMRHIPLAPGSDWRDLPNIEVRLSDGTMARKLRYTHHDRKNGRSSSGALRGVCSCVEAGKACDPAARQFNTLIPWCLPHTGNRHNHWAGLYGRLEWDGFFSTTVTNPEPMGKQGRVLHPEQHRVVSVRECARSQGFPDTYRLFGNILDKHRQVGNAVPPPLAKAIGLEIKLCMLAKARESASAKIKEEEAAKD

SEQ ID NO: 4 - protein, 2377 aa, Cryptococcus neoformans
MTTALTFGGGLFKDNTKFDIDMRGTADGAVNGGNIPNSQSQKRKRASPSPEIESEEDGDDWYEIDYIADSRVIRRKGRQILQYLIHWAGYAVHERTWEDEDGIGGEDCALVQEFYRKNPGKPRLSPSSVRKEVKLARMVEVVITTRRIDGKSRAASSTDQPSPHRLGITSPQANNIGGEDPNPSLTRRPVRSTVSEIAKRPTSKKVHPNKKCKASSDDESDFVFEEGEWDEDEDDDNDVDFRSSEDDEDDEQERSAEEPESDEEIIKPAKKTKSSLPKAKLRPKPANLGGFVTGVRPLNQGLDIKAAVRNMSDDLPPISDIEAMFDHLVSRIPDIVELVRQLNGRKLRVATMCSGTESPLLALNMIAKAIKAQHGLTLAFEHVFSCEIEPFKQAYIERNFTPPILFRDVTELGKKRAHTAYGSMVDVPGDVDILIAGTSCVDYSNLNNVQQDIDANGESGRTFRGMLQWVKKHQPPIVILENVCNAPWDKVVEYFGQIDYDAQYTRLDTKEFYIPHTRTRVYLFATPSSSESDDLPEKWAQTVKDLRRPWSSPFEAFLLHTDDPNIHRARLELASARAQTDGTSRKTTDWNRCESRHQRARQDEALGLLRPLTSWQEAGVCKGLDWTWNDWLLAQTERVVDLLEISTLRMAKDGIDSGFKACIWNVSQNVDRQTGSSKTALAPCLTPNMIPWVTIRGGPVTGREALALQGIPVRELLLTSENEDQLADLAGNAMTTTVVGSAMIAALKVACHKITEGANPEKEAALILEKEAVDDEQVANRIIGEDYLEHHDLDLAKVTKSNLSEILDLACRSSRHCQCEGQSGTAPNILECQECSYRACKSCGGRPEHVYAPCANQRVEPAEFEKRFKGLLPMRVRIAGLTDQCLNAVRKAAEKSNKGSVNDNDWQLWSTALLEGIHDAEFRFRYLKRQSTWTAVYEARRAMLSLVLRNQIPEWRLTIKAPASEPNNSQLRALLLHPVARLQIDIAGQDVLCGPWELCIPSMKTIDIEITGKGELLPSWQASLGLQGPFANTTRWSEVEISLQAEDENTLDRKLSGTYQLLPRCGQAMSSLHKKRPDLSDDGLPQLYFFLDPTRCGESREDRYVFSTSTERLDYGTERPVIARLDSHWREGNEKQRKVKLDVSGAWVKCPEAHLTAIGGDDIAVVANDAAANEIHRDRATFAIPSSASAISASLTTEGCSHAMALLSCRVPLDPTHSESMWRRGAWAEIDLSHQGNTTFANLAWITERLPPLDGLKNWAHIADDVSEHVCERCAPRPPKIHWIKREGKANKKGNKTKSTIIAFEDKLEAGQYEHALKHRPSPFVVQLRLDQDIGSFRIGLNIVSLAHRALSRLPPTTSEHKISLSWRLTPGHVTESPQPRRVFILPSNKQDPENSQPEAFKLPLRKEQLRSLWWMLEQEKATGKTHTFVEEEISESLLPAVGWRAEGKAERPVMVRGGVIADQVGYGKTVISIALVAQTLSLPAPEPATPGLIDLKATLIVVPGHLSKQWPNEIARFTGSMFKVIVIQGMKDLQEKTIAELGKADIIVMASEIFESDVYWSRLEYLSAQPREWLHDTQGGRFFCDRLDAAMESLVSQTKILKEKGSEAAMRAMEDKKKSLVDNVGSKKEVHTAVNFGKRMKGQAYRDKHDSDSKAKPITKEELERWEASEDEDDDENSKTYIPIPKFHSFTGSESIFSASVKKDYKLLPNPVLHMFRFRRVIADEFTYLQKKSLAAVLRLSSSYRWILSGTPPVSDFAAIRSIATFMGIHLGVEDDGEGDVQYQKARAKDQTQAEKFHAFREVHSRAWHNRRDELAQEFLNVFVRQNIAEIEDIPTVEHIHTFKLPASEGAVYLELEHHLQALEMQARKETKFKNVSQGDRNARLEEALSDSKTAEEALLKRCCHFTLDLSDKTQDAKSAQEACDHITSARARQLLACQEDLSRSVNQAIALHGWIKKKGGFSKNDDERQPFAEWIAFSSNISKHQGDIEAARILLKVIEKCGVKDGNIPPSPSDKQSPSIASGAKMDDVKWQLREQTHLLRKLVKELVARVRSLRFFEVVRKIQKGKSDAQIVLESSECGHKPSTNPDIEMAILSCCGHVACHKCMRKAAASQRCVKSGECQAAVRPTNIVKVSSLGVEGELSSGRYGAKLEHLVNLIHSIPKNERVLVFLQWEDLAGKVSEALSAGRIPHVTLSGSAKSRANTLDRFQSTNADSARVLLLKMNDASAAGSNLTTANHAVFLGPLFTNSLFNYRAVETQAIGRVRRYGQQKKVHIHRLLALDTIDMTIFNARRTELKEKTDWEEIPQEEYKGRGSSISMTNEKRTPTLTVKSNPFKRSSSWALASSFRSKKRSMEARDAEGVSDDDENSELSDII

SEQ ID NO: 5 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 6 - protein, 720 aa, Escherichia coli
MDVSYLLDSLNDKQREAVAAPRSNLLVLAGAGSGKTRVLVHRIAWLMSVENCSPYSIMAVTFTNKAAAEMRHRIGQLMGTSQGGMWVGTFHGLAHRLLRAHHMDANLPQDFQILDSEDQLRLLKRLIKAMNLDEKQWPPRQAMWYINSQKDEGLRPHHIQSYGNPVEQTWQKVYQAYQEACDRAGLVDFAELLLRAHELWLNKPHILQHYRERFTNILVDEFQDTNNIQYAWIRLLAGDTGKVMIVGDDDQSIYGWRGAQVENIQRFLNDFPGAETIRLEQNYRSTSNILSAANALIENNNGRLGKKLWTDGADGEPISLYCAFNELDEARFVVNRIKTWQDNGGALAECAILYRSNAQSRVLEEALLQASMPYRIYGGMRFFERQEIKDALSYLRLIANRNDDAAFERVVNTPTRGIGDRTLDVVRQTSRDRQLTLWQACRELLQEKALAGRAASALQRFMELIDALAQETADMPLHVQTDRVIKDSGLRTMYEQEKGEKGQTRIENLEELVTATRQFSYNEEDEDLMPLQAFLSHAALEAGEGQADTWQDAVQLMTLHSAKGLEFPQVFIVGMEEGMFPSQMSLDEGGRLEEERRLAYVGVTRAMQKLTLTYAETRRLYGKEVYHRPSRFIGELPEECVEEVRLRATVSRPVSHQRMGTPMVENDSGYKLGQRVRHAKFGEGTIVNMEGSGEHSRLQVAFQGQGIKWLVAAYARLESV

SEQ ID NO: 7 - protein, 724 aa, Geobacillus stearothermophilus
MNFLSEQLLAHLNKEQQEAVRTTEGPLLIMAGAGSGKTRVLTHRIAYLMAEKHVAPWNILAITFTNKAAREMRERVQSLLGGAAEDVWISTFHSMCVRILRRDIDRIGINRNFSILDPTDQLSVMKTILKEKNIDPKKFEPRTILGTISAAKNELLPPEQFAKRASTYYEKVVSDVYQEYQQRLLRNHSLDFDDLIMTTIQLFDRVPDVLHYYQYKFQYIHIDEYQDTNRAQYTLVKKLAERFQNICAVGDADQSIYRWRGADIQNILSFERDYPNAKVILLEQNYRSTKRILQAANEVIEHNVNRKPKRIWTENPEGKPILYYEAMNEADEAQFVAGRIREAVERGERRYRDFAVLYRTNAQSRVMEEMLLKANIPYQIVGGLKFYDRKEIKDILAYLRVIANPDDDLSLLRIINVPKRGIGASTIDKLVRYAADHELSLFEALGELEMIGLGAKAAGALAAFRSQLEQWTQLQEYVSVTELVEEVLDKSGYREMLKAERTIEAQSRLENLDEFLSVTKHFENVSDDKSLIAFLTDLALISDLDELDGTEQAAEGDAVMLMTLHAAKGLEFPVVFLIGMEEGIFPHNRSLEDDDEMEEERRLAYVGITRAEEELVLTSAQMRTLFGNIQMDPPSRFLNEIPAHLLETASRRQAGASRPAVSRPQASGAVGSWKVGDRANHRKWGIGTVVSVRGGGDDQELDIAFPSPIGIKRLLAKFAPIEKV

SEQ ID NO: 8 - protein, 2002 aa, Homo sapiens
MEQDRTNHVEGNRLSPFLIPSPPICQTEPLATKLQNGSPLPERAHPEVNGDTKWHSFKSYYGIPCMKGSQNSRVSPDFTQESRGYSKCLQNGGIKRTVSEPSLSGLLQIKKLKQDQKANGERRNFGVSQERNPGESSQPNVSDLSDKKESVSSVAQENAVKDFTSFSTHNCSGPENPELQILNEQEGKSANYHDKNIVLLKNKAVLMPNGATVSASSVEHTHGELLEKTLSQYYPDCVSIAVQKTTSHINAINSQATNELSCEITHPSHTSGQINSAQTSNSELPPKPAAVVSEACDADDADNASKLAAMLNTCSFQKPEQLQQQKSVFEICPSPAENNIQGTTKLASGEEFCSGSSSNLQAPGGSSERYLKQNEMNGAYFKQSSVFTKDSFSATTTPPPPSQLLLSPPPPLPQVPQLPSEGKSTLNGGVLEEHHHYPNQSNTTLLREVKIEGKPEAPPSQSPNPSTHVCSPSPMLSERPQNNCVNRNDIQTAGTMTVPLCSEKTRPMSEHLKHNPPIFGSSGELQDNCQQLMRNKEQEILKGRDKEQTRDLVPPTQHYLKPGWIELKAPRFHQAESHLKRNEASLPSILQYQPNLSNQMTSKQYTGNSNMPGGLPRQAYTQKTTQLEHKSQMYQVEMNQGQSQGTVDQHLQFQKPSHQVHFSKTDHLPKAHVQSLCGTRFHFQQRADSQTEKLMSPVLKQHLNQQASETEPFSNSHLLQHKPHKQAAQTQPSQSSHLPQNQQQQQKLQIKNKEEILQTFPHPQSNNDQQREGSFFGQTKVEECFHGENQYSKSSEFETHNVQMGLEEVQNINRRNSPYSQTMKSSACKIQVSCSNNTHLVSENKEQTTHPELFAGNKTQNLHHMQYFPNNVIPKQDLLHRCFQEQEQKSQQASVLQGYKNRNQDMSGQQAAQLAQQRYLIHNHANVFPVPDQGGSHTQTPPQKDTQKHAALRWHLLQKQEQQQTQQPQTESCHSQMHRPIKVEPGCKPHACMHTAPPENKTWKKVTKQENPPASCDNVQQKSIIETMEQHLKQFHAKSLFDHKALTLKSQKQVKVEMSGPVTVLTRQTTAAELDSHTPALEQQTTSSEKTPTKRTAASVLNNFIESPSKLLDTPIKNLLDTPVKTQYDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSSLENSSNKNEKEKSAPSRTKQTENASQAKQLAELLRLSGPVMQQSQQPQPLQKQPPQPQQQQRPQQQQPHHPQTESVNSYSASGSTNPYMRRPNPVSPYPNSSHTSDIYGSTSPMNFYSTSSQAAGSYLNSSNPMNPYPGLLNQNTQYPSYQCNGNLSVDNCSPYLGSYSPQSQPMDLYRYPSQDPLSKLSLPPIHTLYQPRFGNSQSFTSKYLGYGNQNMQGDGFSSCTIRPNVHHVGKLPPYPTHEMDGHFMGATSRLPPNLSNPNMDYKNGEHHSPSHIIHNYSAAPGMFNSSLHALHLQNKENDMLSHTANGLSKMLPALNHDRTACVQGGLHKLSDANGQEKQPLALVQGVASGAEDNDEVWSDSEQSFLDPDIGGVAVAPTHGSILIECAKRELHATTPLKNPNRNHPTRISLVFYQHKSMNEPKHGLALWEAKMAEKAREKEEECEKYGPDYVPQKSHGKKVKREPAEPHETSEPTYLRFIKSLAERTMSVTTDSTVTTSPYAFTRVTGPYNRYI

SEQ ID NO: 9 - protein, 1912 aa, Mus musculus
MEQDRTTHAEGTRLSPFLIAPPSPISHTEPLAVKLQNGSPLAERPHPEVNGDTKWQSSQSCYGISHMKGSQSSHESPHEDRGYSRCLQNGGIKRTVSEPSLSGLHPNKILKLDQKAKGESNIFEESQERNHGKSSRQPNVSGLSDNGEPVTSTTQESSGADAFPTRNYNGVEIQVLNEQEGEKGRSVTLLKNKIVLMPNGATVSAHSEENTRGELLEKTQCYPDCVSIAVQSTASHVNTPSSQAAIELSHEIPQPSLTSAQINFSQTSSLQLPPEPAAMVTKACDADNASKPAIVPGTCPFQKAEHQQKSALDIGPSRAENKTIQGSMELFAEEYYPSSDRNLQASHGSSEQYSKQKETNGAYFRQSSKFPKDSISPTTVTPPSQSLLAPRLVLQPPLEGKGALNDVALEEHHDYPNRSNRTLLREGKIDHQPKTSSSQSLNPSVHTPNPPLMLPEQHQNDCGSPSPEKSRKMSEYLMYYLPNHGHSGGLQEHSQYLMGHREQEIPKDANGKQTQGSVQAAPGWIELKAPNLHEALHQTKRKDISLHSVLHSQTGPVNQMSSKQSTGNVNMPGGFQRLPYLQKTAQPEQKAQMYQVQVNQGPSPGMGDQHLQFQKALYQECIPRTDPSSEAHPQAPSVPQYHFQQRVNPSSDKHLSQQATETQRLSGFLQHTPQTQASQTPASQNSNFPQICQQQQQQQLQRKNKEQMPQTFSHLQGSNDKQREGSCFGQIKVEESFCVGNQYSKSSNFQTHNNTQGGLEQVQNINKNFPYSKILTPNSSNLQILPSNDTHPACEREQALHPVGSKTSNLQNMQYFPNNVTPNQDVHRCFQEQAQKPQQASSLQGLKDRSQGESPAPPAEAAQQRYLVHNEAKALPVPEQGGSQTQTPPQKDTQKHAALRWLLLQKQEQQQTQQSQPGHNQMLRPIKTEPVSKPSSYRYPLSPPQENMSSRIKQEISSPSRDNGQPKSIIETMEQHLKQFQLKSLCDYKALTLKSQKHVKVPTDIQAAESENHARAAEPQATKSTDCSVLDDVSESDTPGEQSQNGKCEGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKCGICTNRRCSQNETRNCCCQGENPETCGASFSFGCSWSMYYNGCKFARSKKPRKFRLHGAEPKEEERLGSHLQNLATVIAPIYKKLAPDAYNNQVEFEHQAPDCCLGLKEGRPFSGVTACLDFSAHSHRDQQNMPNGSTVVVTLNREDNREVGAKPEDEQFHVLPMYIIAPEDEFGSTEGQEKKIRMGSIEVLQSFRRRRVIRIGELPKSCKKKAEPKKAKTKKAARKRSSLENCSSRTEKGKSSSHTKLMENASHMKQMTAQPQLSGPVIRQPPTLQRHLQQGQRPQQPQPPQPQPQTTPQPQPQPQHIMPGNSQSVGSHCSGSTSVYTRQPTPHSPYPSSAHTSDIYGDTNHVNFYPTSSHASGSYLNPSNYMNPYLGLLNQNNQYAPFPYNGSVPVDNGSPFLGSYSPQAQSRDLHRYPNQDHLTNQNLPPIHTLHQQTFGDSPSKYLSYGNQNMQRDAFTTNSTLKPNVHHLATFSPYPTPKMDSHFMGAASRSPYSHPHTDYKTSEHHLPSHTIYSYTAAASGSSSSHAFHNKENDNIANGLSRVLPGFNHDRTASAQELLYSLTGSSQEKQPEVSGQDAAAVQEIEYWSDSEHNFQDPCIGGVAIAPTHGSILIECAKCEVHATTKVNDPDRNHPTRISLVLYRHKNLFLPKHCLALWEAKMAEKARKEEECGKNGSDHVSQKNHGKQEKREPTGPQEPSYLRFIQSLAENTGSVTTDSTVTTSPYAFTQVTGPYNTFV

(19) United States
(12) Patent Application Publication
(10) Pub. No.: US 2026/0201443 A1
(43) Pub. Date: Jul. 16, 2026

(54) USE OF 5-HYDROXYMETHYCYTOSINE OR 5-METHYLCYTOSINE FOR DISEASE DETECTION

(71) Applicant: Biomodal Limited, Essex (GB)

(72) Inventors: Shankar Balasubramanian, Cambridge (GB); Páidí Creed, Cambridge (GB)

(21) Appl. No.: 19/552,942

(22) Filed: Feb. 27, 2026

Related U.S. Application Data
(63) Continuation of application No. PCT/US2025/051855, filed on Oct. 21, 2025.
(60) Provisional application No. 63/711,035, filed on Oct. 23, 2024.
(60) Provisional application No. 63/713,178, filed on Oct. 29, 2024.

(51) Int. Cl. C12Q 1/6806; C12Q 1/6869

(52) U.S. Cl. CPC C12Q 1/6806; C12Q 1/6869

(57) ABSTRACT

Provided herein are methods and systems related to obtaining and processing sequence reads. The methods may comprise obtaining cell-free DNA sequences from a subject and obtaining sequencing reads from the cell-free DNA sequences that identify cell-free DNA sequences, 5-methylcytosines, and 5-hydroxymethylcytosines. The methods may involve applying a trained classifier to the sequencing reads.

### CROSS-REFERENCE

This application is a continuation of International Application No. PCT/US2025/051855, filed Oct. 21, 2025, which claims priority to U.S. Provisional Patent Application No. 63/711,035, filed Oct. 23, 2024, and U.S. Provisional Patent Application No. 63/713,178, filed Oct. 29, 2024, which are entirely incorporated herein by reference.

### SEQUENCE LISTING

The instant application contains a Sequence Listing which has been submitted electronically in XML format and is hereby incorporated by reference in its entirety. Said XML copy, created on Nov. 5, 2025, is named 47224-756_301_SL.xml and is 18,858 bytes in size.

### BACKGROUND

The most common form of epigenetic modification is DNA methylation, which is brought about by covalent addition of the methyl group at the fifth carbon of the cytosine ring, resulting in 5-methylcytosine (5mC), which is catalyzed by DNA methyltransferases. 5mC can be converted to 5-hydroxymethylcytosine (5hmC) in mammalian DNA by the ten-eleven translocation (TET) enzymes.

### SUMMARY

In some aspects, the present disclosure provides for a method of processing cell-free deoxyribonucleic acid (cfDNA), comprising: (a) obtaining cfDNA sequences from a sample obtained from a subject, wherein said cfDNA sequences are from regions across a genome where differential methylation is indicative of cancer status; (b) obtaining sequencing reads corresponding to said cfDNA sequences that identify 5-hydroxymethylcytosine (5hmC) in said cfDNA sequences; (c) identifying, from said sequencing reads, differences in 5hmC abundance in said regions across said genome when compared to 5hmC abundance in said regions across said genome measured in a healthy subject; and (d) generating a report indicating an early-stage cancer status in said subject based at least in part on said differences in 5hmC abundance. In some embodiments, said early-stage cancer status is indicated based at least in part on an increase in 5hmC abundance. In some embodiments, said early-stage cancer status is a stage I cancer status. In some embodiments, the method further comprises detecting an early-stage cancer status in said subject using at least said differences. In some embodiments, said regions across said genome have been identified by comparing levels of 5-methylcytosine (5mC) in a sample(s) obtained from a healthy subject(s) and a sample(s) obtained from a late-stage cancer patient. In some embodiments, said regions across said genome have been identified by comparing levels of 5-methylcytosine (5mC) in a sample(s) obtained from a healthy subject(s) and a sample(s) obtained from an early-stage cancer patient(s). In some embodiments, said regions across said genome have been identified by comparing levels of 5-methylcytosine (5mC) in a sample(s) obtained from a healthy subject(s), a sample(s) obtained from an early-stage cancer patient(s), and a sample(s) obtained from a late-stage cancer patient (s). In some embodiments, said early-stage cancer status is of a cancer selected from the group consisting of colorectal cancer, gastric cancer, lung cancer, liver cancer, ovarian cancer, breast cancer, prostate cancer, esophageal cancer, thyroid cancer, uterine cancer, pancreatic cancer, bladder cancer, hematological cancer, brain cancer, and kidney cancer. In some embodiments, said method of obtaining sequencing reads in (b) is selected from the group consisting of short read sequencing and long read sequencing. In some embodiments, said short read sequencing is sequencing by synthesis. In some embodiments, said long read sequencing is nanopore sequencing or single molecule real-time sequencing. In some embodiments, said method is capable of identifying an early-stage cancer status in said subject with a sensitivity of at least about 70%, at least about 75%, at least about 80%, at least about 85%, at least about 90%, at least about 95%, at least about 97%, at least about 99%, or at least about 99.9%. In some embodiments, said method is capable of identifying an early-stage cancer status in said subject with a specificity of at least about 30%, at least about 40%, at least about 50%, at least about 60%, at least about 70%, at least about 80%, at least about 90%, at least about 95%, at least about 97%, at least about 99%, or at least about 99.9%. In some embodiments, said method is capable of identifying an early-stage cancer status in said subject with a sensitivity of at least about 80% and a specificity of at least about 30%, at least about 40%, at least about 50%, at least about 60%, at least about 70%, at least about 80%, at least about 90%, at least about 95%, at least about 97%, at least about 99%, or at least about 99.9%. In some embodiments, said method is capable of identifying an early-stage cancer status in said subject with an accuracy of at least about 70%, at least about 75%, at least about 80%, at least about 85%, at least about 90%, at least about 95%, at least about 97%, at least about 99%, or at least about 99.9%. In some embodiments, said sequencing reads corresponding to said cfDNA sequences identify 5-methylcytosine (5mC) in said cfDNA sequences. In some embodiments, said sequencing reads differences in 5mC abundance in said regions across said genome when compared to 5mC abundance in said regions across said genome measured in a healthy subject. In some embodiments, the method further comprises generating a report indicating said early-stage cancer status in said subject using at least said differences in 5hmC abundance and said differences in said 5mC abundance. In some embodiments, the method further comprises performing a next generation sequencing reaction to obtain said sequencing reads. In some embodiments, the method further comprises attaching a hairpin to strands of said cfDNA sequences such that said strands are covalently linked from 5′ to 3′, thereby producing hairpin-linked cfDNA sequences. In some embodiments, the method further comprises digesting said hairpin-linked cfDNA sequences to generate hairpin-linked strands of said cfDNA sequences. In some embodiments, the method further comprises extending said harpin-linked strands of said cfDNA sequences to produce double-stranded hairpin-linked cfDNA sequences. In some embodiments, the method further comprises adding a non-deaminatable moiety to 5hmC of said double-stranded hairpin-linked cfDNA sequences. In some embodiments, said adding said non-deaminatable moiety to said 5hmC of said double-stranded hairpin-linked cfDNA sequences comprises adding a sugar residue, a glucose molecule, or a glucose-derivative donor substrate to said 5hmC of said double-stranded hairpin-linked cfDNA sequences. In some embodiments, said adding said glucose molecule or said glucose-derivative donor substrate to said 5hmC of said double-stranded hairpin-linked cfDNA sequences comprises contacting said double-stranded hairpin-linked cfDNA sequences with an α-glucosyltransferase (AGT) polypeptide, a β-glucosyltransferase (BGT) polypeptide, or a catalytically active fragment thereof. In some embodiments, the method further comprises contacting said double-stranded hairpin-linked cfDNA sequences with a DNA methyltransferase. In some embodiments, said DNA methyltransferase comprises an amino acid sequence that is at least 90% identical to DNMT1 or a catalytically active fragment thereof, or at least 90° % identical to DNMT5 or a catalytically active fragment thereof. In some embodiments, the method further comprises contacting said double-stranded hairpin-linked cfDNA fragment with a dioxygenase and a glycosyltransferase. In some embodiments, said dioxygenase comprises a TET enzyme or fragment thereof. In some embodiments, said glycosyltransferase comprises an α-glucosyltransferase (AGT) polypeptide, a β-glucosyltransferase (BGT) polypeptide, or a catalytically active fragment thereof. In some embodiments, the method further comprises deaminating said double-stranded hairpin-linked cfDNA sequences. In some embodiments, said deaminating comprises contacting said double-stranded hairpin-linked cfDNA sequences with an apolipoprotein B mRNA editing enzyme, catalytic polypeptide-like (APOBEC) enzyme, or a catalytically active fragment thereof. In some embodiments, said deaminating further comprises contacting said double-stranded hairpin-linked cfDNA sequences with said APOBEC enzyme or said catalytically active fragment of said APOBEC enzyme in the presence of a helicase. In some embodiments, said helicase comprises an amino acid sequence that is at least 90% identical to a UvrD helicase, a Geobacillus sterothermophilus Bad protein, a PcrA helicase, or a catalytically active fragment thereof. In some embodiments, the method further comprises performing a next-generation sequencing reaction on said double-stranded hairpin-linked cfDNA sequences. In some embodiments, (c) further comprises identifying differences in 5hmC abundance in at least about 1,000, at least about 2,500, at least about 5,000; at least about 7,500, at least about 10,000, or at least about 11,000 of said regions across said genome. In some embodiments, the method further comprises administering or recommending administration of a chemotherapeutic agent to said subject based at least in part on said early-stage cancer status.

In some aspects, the present disclosure provides for a method of processing cfDNA from a subject having or suspected of having cancer, comprising: (a) obtaining cell-free DNA (cfDNA) sequences from a subject; (b) obtaining sequencing reads from said cfDNA sequences that identify: (i) said cfDNA sequences; (ii) 5-methylcytosine (5mC) in said cfDNA sequences; and (iii) 5-hydroxymethylcytosine (5hmC) in said cfDNA sequences; and (c) applying a trained classifier to said sequencing reads, wherein said trained classifier has been trained on location or abundance information for 5mC and 5hmC in cfDNA sequences indicative of cancer status. In some embodiments, said cfDNA sequences are obtained from a plasma, serum, or whole blood sample from said subject. In some embodiments, said trained classifier has been trained on location information or abundance information for 5mC and 5hmC in cfDNA sequences indicative of cancer status for a cancer selected from the group consisting of colorectal cancer, gastric cancer, lung cancer, liver cancer, ovarian cancer, breast cancer, prostate cancer, esophageal cancer, thyroid cancer, uterine cancer, pancreatic cancer, bladder cancer, hematological cancer, brain cancer, and kidney cancer. In some embodiments, said trained classifier has been trained on location information or abundance information for 5mC and 5hmC in cfDNA sequences indicative of cancer stages I, II, III or IV, or combinations thereof. In some embodiments, said cancer stage is stage I. In some embodiments, said trained classifier is capable of performing a classification between said subject not having cancer and said subject having cancer with at least about 80%, at least about 85%, at least about 90%, at least about 95%, at least about 97%, at least about 99%, or at least about 99.9% sensitivity. In some embodiments, said trained classifier is capable of performing a classification between said subject not having cancer and said subject having cancer with at least about 30%, at least about 40%, at least about 50%, at least about 60%, at least about 70%, at least about 80%, at least about 90%, at least about 95%, at least about 97%, at least about 99%, or at least about 99.9% specificity. In some embodiments, said trained classifier is capable of performing a classification between said subject not having cancer and said subject having cancer with an accuracy of at least about 70%, at least about 75%, at least about 80%, at least about 85%, at least about 90%, at least about 95%, at least about 97%, at least about 99%, or at least about 99.9%. In some embodiments, said trained classifier is capable of performing a classification between said subject not having cancer and said subject having an early-stage cancer with a sensitivity of at least about 80%, at least about 85%, at least about 90%, at least about 95%, at least about 97%, at least about 99%, or at least about 99.9%. In some embodiments, said trained classifier is capable of performing a classification between said subject not having cancer and said subject having an early-stage cancer with a specificity of at least about 30%, at least about 40%, at least about 50%, at least about 60%, at least about 70%, at least about 80%, at least about 90%, at least about 95%, at least about 97%, at least about 99%, or at least about 99.9%. In some embodiments, said trained classifier is capable of performing a classification between said subject not having cancer and said subject having an early-stage cancer with a sensitivity of at least about 70%, at least about 75%, at least about 80% and a specificity of at least about 30%, at least about 40%, at least about 50%, at least about 60%, at least about 70%, at least about 80%, at least about 90%, at least about 95%, at least about 97%, at least about 99%, or at least about 99.9%. In some embodiments, said trained classifier is capable of performing a classification between said subject not having cancer and said subject having an early-stage cancer with an accuracy of at least about 70%, at least about 75%, at least about 80%, at least about 85%, at least about 90%, at least about 95%, at least about 97%, at least about 99%, or at least about 99.9%. In some embodiments, said trained classifier is capable of performing a classification between said subject not having cancer and said subject having a late-stage cancer with a sensitivity of at least about 70%, at least about 75%, at least about 80%, at least about 85%, at least about 90%, at least about 95%, at least about 97%, at least about 99%, or at least about 99.9%. In some embodiments, said trained classifier is capable of performing a classification between said subject not having cancer and said subject having a late-stage cancer with a specificity of at least about 30%, at least about 40%, at least about 50%, at least about 60%, at least about 70%, at least about 80%, at least about 90%, at least about 95%, at least about 97%, at least about 99%, or at least about 99.9%. In some embodiments, said trained classifier is capable of performing a classification between said subject not having cancer and said subject having a late-stage cancer with a sensitivity of at least about 80% and a specificity of at least about 30%, at least about 40%, at least about 50%, at least about 60%, at least about 70%, at least about 80%, at least about 90%, at least about 95%, at least about 97%, at least about 99%, or at least about 99.9%. In some embodiments, said trained classifier is capable of performing a classification between said subject not having cancer and said subject having a late-stage cancer with an accuracy of at least about 70%, at least about 75%, at least about 80%, at least about 85%, at least about 90%, at least about 95%, at least about 97%, at least about 99%, or at least about 99.9%. In some embodiments, the method further comprises attaching a hairpin to strands of said cfDNA sequences such that said strands are covalently linked from 5′ to 3′, thereby producing hairpin-linked cfDNA sequences. In some embodiments, the method further comprises digesting said hairpin-linked cfDNA sequences to generate hairpin-linked strands of said cfDNA sequences. In some embodiments, the method further comprises extending said harpin-linked strands of said cfDNA sequences to produce double-stranded hairpin-linked cfDNA sequences. In some embodiments, the method further comprises adding a non-deaminatable moiety to 5hmC of said double-stranded hairpin-linked cfDNA sequences. In some embodiments, said adding said non-deaminatable moiety to said 5hmC of said double-stranded hairpin-linked cfDNA sequences comprises adding a sugar residue, a glucose molecule, or a glucose-derivative donor substrate to said 5hmC of said double-stranded hairpin-linked cfDNA sequences. In some embodiments, said adding said glucose molecule or said glucose-derivative donor substrate to said 5hmC of said double-stranded hairpin-linked cfDNA sequences comprises contacting said double-stranded hairpin-linked cfDNA sequences with an α-glucosyltransferase (AGT) polypeptide, a β-glucosyltransferase (BGT) polypeptide, or a catalytically active fragment thereof. In some embodiments, the method further comprises contacting said double-stranded hairpin-linked cfDNA sequences with a DNA methyltransferase. In some embodiments, said DNA methyltransferase comprises an amino acid sequence that is at least 90% identical to DNMT1 or a catalytically active fragment thereof, or at least 90% identical to DNMT5 or a catalytically active fragment thereof. In some embodiments, the method further comprises contacting said double-stranded hairpin-linked cfDNA sequences with a dioxygenase and a glycosyltransferase. In some embodiments, said dioxygenase comprises a TET enzyme or fragment thereof. In some embodiments, said glycosyltransferase comprises an α-glucosyltransferase (AGT) polypeptide, a β-glucosyltransferase (BGT) polypeptide, or a catalytically active fragment thereof. In some embodiments, the method further comprises deaminating said double-stranded hairpin-linked cfDNA sequences. In some embodiments, said deaminating comprises contacting said double-stranded hairpin-linked cfDNA sequences with an apolipoprotein B mRNA editing enzyme, catalytic polypeptide-like (APOBEC) enzyme, or a catalytically active fragment thereof. In some embodiments, said deaminating further comprises contacting said double-stranded hairpin-linked cfDNA sequences with said APOBEC enzyme or said catalytically active fragment of said APOBEC enzyme in the presence of a helicase. In some embodiments, said helicase comprises an amino acid sequence that is at least 90% identical to a UvrD helicase, a Geobacillus sterothermophilus Bad protein, a PcrA helicase, or a catalytically active fragment thereof. In some embodiments, the method further comprises performing a next-generation sequencing reaction on said double-stranded hairpin-linked cfDNA sequences. In some embodiments, the method further comprises performing a next generation sequencing reaction to obtain said sequencing reads. In some embodiments, the method further comprises detecting cells of said cancer or absence thereof in said subject using at least said applying said trained classifier to said sequencing reads. In some embodiments, the method further comprises administering or recommending administration of a chemotherapeutic agent to said subject based at least in part on said detecting said cells of said cancer in said subject. In some embodiments, said location or abundance information comprises locations or abundances of 5mC or 5hmC bases in differentially methylated regions (DMRs) of said cfDNA sequences, wherein said DMRs are differentially methylated in a genome of a subject having said cancer when compared to a genome of said subject not having said cancer. In some embodiments, said location or abundance information comprises locations or abundances of said 5mC or said 5hmC bases in at least about 1,000, at least about 2,500, at least about 5,000; at least about 7,500, at least about 10,000, or at least about 11,000 of said DMRs from said cfDNA sequences. In some embodiments, the method further comprises obtaining sequence reads from at least about 1,000, at least about 2,500, at least about 5,000, at least about 7,500, at least about 10,000, or at least about 11,000 DMRs from said cfDNA sequences. In some embodiments, the method further comprises generating a report indicating a stage I, II, III or IV cancer status in said subject using at least said trained classifier.

In some aspects, the present disclosure provides for a method of processing cell-free deoxyribonucleic acid (cfDNA), comprising: (a) obtaining cfDNA sequences from a sample obtained from a subject, wherein said cfDNA sequences are from regions across a genome where differential methylation is indicative of cancer status; (b) obtaining sequencing reads corresponding to said cfDNA sequences that identify 5-hydroxymethylcytosine (5hmC) in said cfDNA sequences; (c) identifying, from said sequencing reads, differences in 5hmC abundance in said regions across said genome when compared to 5hmC abundance in said regions across said genome measured in a healthy subject; and (d) providing a therapeutic intervention to said subject based at least in part on said difference. In some embodiments, said therapeutic intervention comprises a chemotherapeutic agent. In some embodiments, said regions across said genome have been identified by comparing levels of 5-methylcytosine (5mC) in a sample(s) obtained from a healthy subject(s) and a sample(s) obtained from a late-stage cancer patient. In some embodiments, said regions across said genome have been identified by comparing levels of 5-methylcytosine (5mC) in a sample(s) obtained from a healthy subject(s) and a sample(s) obtained from an early-stage cancer patient(s). In some embodiments, said regions across said genome have been identified by comparing levels of 5-methylcytosine (5mC) in a sample(s) obtained from a healthy subject(s), a sample(s) obtained from an early-stage cancer patient(s), and a sample(s) obtained from a late-stage cancer patient (s). In some embodiments, said method of obtaining sequencing reads in (b) is selected from the group consisting of short read sequencing and long read sequencing. In some embodiments, said short read sequencing is sequencing by synthesis. In some embodiments, said long read sequencing is nanopore sequencing or single molecule real-time sequencing. In some embodiments, said method is capable of identifying an early-stage cancer status in said subject with a sensitivity of at least about 70%, at least about 75%, at least about 80%, at least about 85%, at least about 90%, at least about 95%, at least about 97%, at least about 99%, or at least about 99.9%. In some embodiments, said method is capable of identifying an early-stage cancer status in said subject with a specificity of at least about 30%, at least about 40%, at least about 50%, at least about 60%, at least about 70%, at least about 80%, at least about 90%, at least about 95%, at least about 97%, at least about 99%, or at least about 99.9%. In some embodiments, said method is capable of identifying an early-stage cancer status in said subject with a sensitivity of at least about 70%, at least about 75%, at least about 80% and a specificity of at least about 30%, at least about 40%, at least about 50%, at least about 60%, at least about 70%, at least about 80%, at least about 90%, at least about 95%, at least about 97%, at least about 99%, or at least about 99.9%. In some embodiments, said method is capable of identifying an early-stage cancer status in said subject with an accuracy of at least about 70%, at least about 75%, at least about 80%, at least about 85%, at least about 90%, at least about 95%, at least about 97%, at least about 99%, or at least about 99.9%. In some embodiments, said sequencing reads corresponding to said cfDNA sequences identify 5-methylcytosine (5mC) in said cfDNA sequences. In some embodiments, the method further comprises identifying from said sequencing reads differences in 5mC abundance in said regions across said genome when compared to 5mC abundance in said regions across said genome measured in a healthy subject. In some embodiments, the method further comprises attaching a hairpin to strands of said cfDNA sequences such that said strands are covalently linked from 5′ to 3′, thereby producing hairpin-linked cfDNA sequences. In some embodiments, the method further comprises digesting said hairpin-linked cfDNA sequences to generate hairpin-linked strands of said cfDNA sequences. In some embodiments, the method further comprises extending said harpin-linked strands of said cfDNA sequences to produce double-stranded hairpin-linked cfDNA sequences. In some embodiments, the method further comprises adding a non-deaminatable moiety to 5hmC of said double-stranded hairpin-linked cfDNA sequences. In some embodiments, said adding said non-deaminatable moiety to said 5hmC of said double-stranded hairpin-linked cfDNA sequences comprises adding a sugar residue, a glucose molecule, or a glucose-derivative donor substrate to said 5hmC of said double-stranded hairpin-linked cfDNA sequences. In some embodiments, said adding said glucose molecule or said glucose-derivative donor substrate to said 5hmC of said double-stranded hairpin-linked cfDNA sequences comprises contacting said double-stranded hairpin-linked cfDNA sequences with an α-glucosyltransferase (AGT) polypeptide, a β-glucosyltransferase (BGT) polypeptide, or a catalytically active fragment thereof. In some embodiments, the method further comprises contacting said double-stranded hairpin-linked cfDNA sequences with a DNA methyltransferase. In some embodiments, said DNA methyltransferase comprises an amino acid sequence that is at least 90% identical to DNMT1 or a catalytically active fragment thereof, or at least 90% identical to DNMT5 or a catalytically active fragment thereof. In some embodiments, the method further comprises contacting said double-stranded hairpin-linked cfDNA fragment with a dioxygenase and a glycosyltransferase. In some embodiments, said dioxygenase comprises a TET enzyme or fragment thereof. In some embodiments, said glycosyltransferase comprises an α-glucosyltransferase (AGT) polypeptide, a β-glucosyltransferase (BGT) polypeptide, or a catalytically active fragment thereof. In some embodiments, the method further comprises deaminating said double-stranded hairpin-linked cfDNA sequences. In some embodiments, said deaminating comprises contacting said double-stranded hairpin-linked cfDNA sequences with an apolipoprotein B mRNA editing enzyme, catalytic polypeptide-like (APOBEC) enzyme, or a catalytically active fragment thereof. In some embodiments, said deaminating further comprises contacting said double-stranded hairpin-linked cfDNA sequences with said APOBEC enzyme or said catalytically active fragment of said APOBEC enzyme in the presence of a helicase. In some embodiments, said helicase comprises an amino acid sequence that is at least 90% identical to a UvrD helicase, a Geobacillus sterothermophilus Bad protein, a PcrA helicase, or a catalytically active fragment thereof. In some embodiments, the method further comprises performing a next-generation sequencing reaction on said double-stranded hairpin-linked cfDNA sequences.

In some aspects, the present disclosure provides for a method of processing cfDNA from a subject having or suspected of having cancer, comprising: (a) obtaining cell-free DNA (cfDNA) sequences from a subject; (b) obtaining sequencing reads from said cfDNA sequences that identify: (i) said cfDNA sequences; (ii) 5-methylcytosine (5mC) in said cfDNA sequences; and (iii) 5-hydroxymethylcytosine (5hmC) in said cfDNA sequences; (c) applying a trained classifier to said sequencing reads, wherein said trained classifier has been trained on location or abundance information for 5mC and 5hmC in cfDNA sequences indicative of cancer status; and (d) providing a diagnostic assessment of said subject based at least in part on said applying said trained classifier to said sequencing reads. In some embodiments, said diagnostic assessment is provided by a healthcare provider. In some embodiments, the method further comprises providing a therapeutic intervention to said subject based at least in part on said diagnostic assessment. In some embodiments, said therapeutic intervention comprises a chemotherapeutic agent. In some embodiments, said trained classifier has been trained on location information or abundance information for 5mC and 5hmC in cfDNA sequences indicative of cancer status for a cancer selected from the group consisting of colorectal cancer, gastric cancer, lung cancer, liver cancer, ovarian cancer, breast cancer, prostate cancer, esophageal cancer, thyroid cancer, uterine cancer, pancreatic cancer, bladder cancer, hematological cancer, brain cancer, and kidney cancer. In some embodiments, said trained classifier has been trained on location information or abundance information for 5mC and 5hmC in cfDNA sequences indicative of cancer stages I, II, III or IV, or combinations thereof. In some embodiments, said trained classifier is capable of performing a classification between said subject not having cancer and said subject having cancer with at least about 80%, at least about 85%, at least about 90%, at least about 95%, at least about 97%, at least about 99%, or at least about 99.9% sensitivity. In some embodiments, said trained classifier is capable of performing a classification between said subject not having cancer and said subject having cancer with at least about 30%, at least about 40%, at least about 50%, at least about 60%, at least about 70%, at least about 80%, at least about 90%, at least about 95%, at least about 97%, at least about 99%, or at least about 99.9% specificity. In some embodiments, said trained classifier is capable of performing a classification between said subject not having cancer and said subject having cancer with an accuracy of at least about 70%, at least about 75%, at least about 80%, at least about 85%, at least about 90%, at least about 95%, at least about 97%, at least about 99%, or at least about 99.9%. In some embodiments, the method further comprises attaching a hairpin to strands of said cfDNA sequences such that said strands are covalently linked from 5′ to 3′, thereby producing hairpin-linked cfDNA sequences. In some embodiments, the method further comprises digesting said hairpin-linked cfDNA sequences to generate hairpin-linked strands of said cfDNA sequences. In some embodiments, the method further comprises extending said harpin-linked strands of said cfDNA sequences to produce double-stranded hairpin-linked cfDNA sequences. In some embodiments, the method further comprises adding a non-deaminatable moiety to 5hmC of said double-stranded hairpin-linked cfDNA sequences. In some embodiments, said adding said non-deaminatable moiety to said 5hmC of said double-stranded hairpin-linked cfDNA sequences comprises adding a sugar residue, a glucose molecule, or a glucose-derivative donor substrate to said 5hmC of said double-stranded hairpin-linked cfDNA sequences. In some embodiments, said adding said glucose molecule or said glucose-derivative donor substrate to said 5hmC of said double-stranded hairpin-linked cfDNA sequences comprises contacting said double-stranded hairpin-linked cfDNA sequences with an α-glucosyltransferase (AGT) polypeptide, a β-glucosyltransferase (BGT) polypeptide, or a catalytically active fragment thereof. In some embodiments, the method further comprises contacting said double-stranded hairpin-linked cfDNA sequences with a DNA methyltransferase. In some embodiments, said DNA methyltransferase comprises an amino acid sequence that is at least 90% identical to DNMT1 or a catalytically active fragment thereof, or at least 90% identical to DNMT5 or a catalytically active fragment thereof. In some embodiments, the method further comprises contacting said double-stranded hairpin-linked cfDNA fragment with a dioxygenase and a glycosyltransferase. In some embodiments, said dioxygenase comprises a TET enzyme or fragment thereof. In some embodiments, said glycosyltransferase comprises an α-glucosyltransferase (AGT) polypeptide, a β-glucosyltransferase (BGT) polypeptide, or a catalytically active fragment thereof. In some embodiments, the method further comprises deaminating said double-stranded hairpin-linked cfDNA sequences. In some embodiments, said deaminating comprises contacting said double-stranded hairpin-linked cfDNA sequences with an apolipoprotein B mRNA editing enzyme, catalytic polypeptide-like (APOBEC) enzyme, or a catalytically active fragment thereof. In some embodiments, said deaminating further comprises contacting said double-stranded hairpin-linked cfDNA sequences with said APOBEC enzyme or said catalytically active fragment of said APOBEC enzyme in the presence of a helicase. In some embodiments, said helicase comprises an amino acid sequence that is at least 90% identical to a UvrD helicase, a Geobacillus sterothermophilus Bad protein, a PcrA helicase, or a catalytically active fragment thereof. In some embodiments, the method further comprises performing a next-generation sequencing reaction on said double-stranded hairpin-linked cfDNA sequences.

In some aspects, the present disclosure provides for a method of processing cell-free deoxyribonucleic acid (cfDNA), comprising: (a) obtaining cfDNA sequences from a sample obtained from a subject, wherein said cfDNA sequences are from regions across a genome where differential methylation is indicative of cancer status; (b) obtaining sequencing reads corresponding to said cfDNA sequences that identify 5-hydroxymethylcytosine (5hmC) and 5-methylcytosine (5mC) in said cfDNA sequences; (c) identifying, from said sequencing reads, differences in 5hmC abundance and differences in 5mC abundance in said regions across said genome when compared to 5hmC abundance and 5mC abundance in said regions across said genome measured in a healthy subject; and (d) generating a report indicating an early-stage cancer status in said subject based at least in part on said differences in 5hmC abundance and said differences in 5mC abundance.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### COLOR DRAWINGS

The patent or application file contains at least one drawing executed in color. Copies of this patent or patent application publication with color drawing(s) will be provided by the office upon request and payment of the necessary fee.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

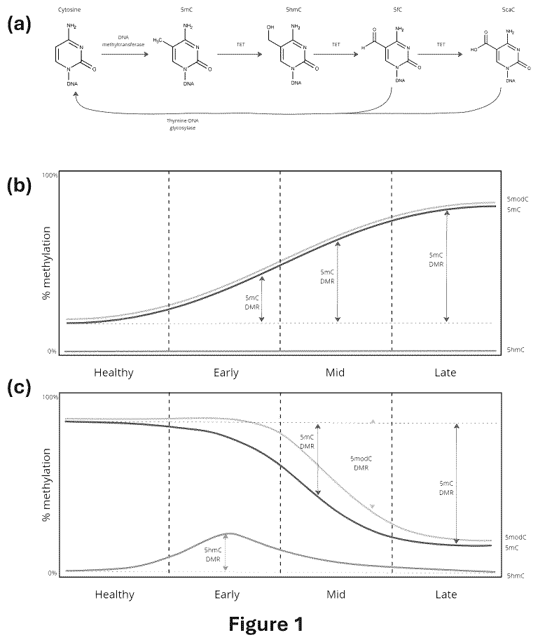

FIG. 1 depicts dynamics of cytosine methylation in mammals. Panel (a) shows a schematic of the enzymatic operations involved in cytosine methylation and demethylation. DNA methyltransferases catalyse the addition of a methyl group to a cytosine base in a CpG context. The methyl group is oxidised by TET enzymes to 5-hydroxymethylcytosine (5hmC), and downstream oxidised derivatives 5-formylcytosine (5fC) and 5-carboxylcytosine (5caC), before thymine DNA glycosylase mediates the removal of 5fC or 5caC, with subsequent replacement with unmodified cytosine by gap-filling repair. Panel (b) Schematic representation of the increase in 5mC leading to hypermethylation or (c) loss of 5mC leading to hypomethylation in a region of the genome. Panel (b) represents a region with 5mC hypermethylation in late-stage cancer, where 5mC accumulates during disease progression. For these regions, changes in 5mC and modC are approximately equivalent and can therefore be expected to have similar power as biomarkers to distinguish between disease stages and healthy individuals. Panel (c) represents a region with hypomethylation in late-stage disease. Changes in 5hmC are proportionally larger at the early stage than changes in 5mC or modC. At later stages, as demethylation completes, 5mC and modC become proportionally larger. Changes in modC are masked by the conflation of 5mC and 5hmC and become distinguishable at mid- to late-stage.

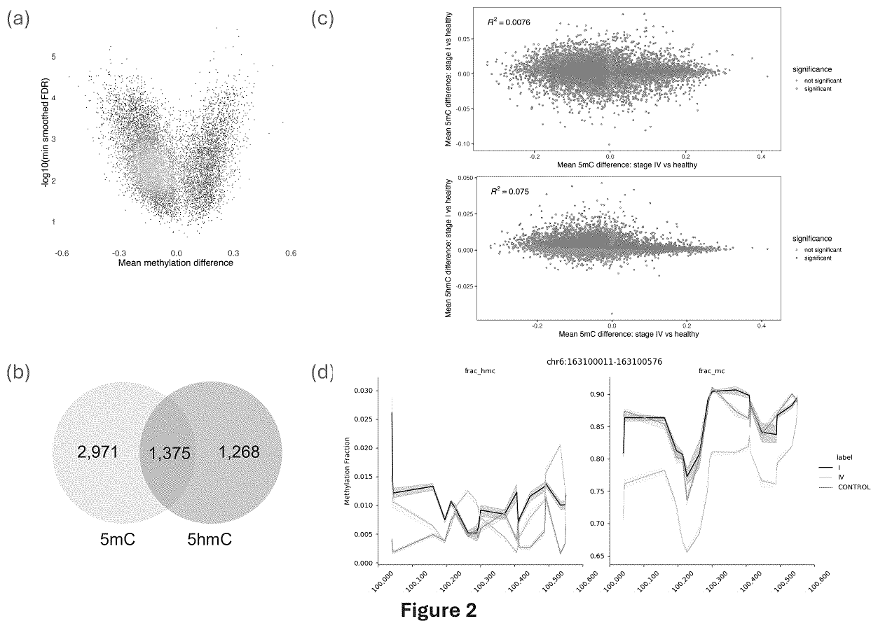

FIG. 2 depicts dynamics of 5mC and 5hmC in CRC samples as analyzed herein. Panel (a) shows a Volcano plot for differentially methylated regions (DMRs) between stage IV CRC tissue and adjacent matched normal, using data from TCGA. Colour indicates density of points, with yellow used to indicate a higher density. Panel (b) shows a Venn diagram of regions with statistically significant (q<0.05) differences in 5mC and 5hmC in stage I plasma. Panel (c) -shows a scatter plot comparing 5mC (upper) and 5hmc (lower) differences measured in stage I and stage IV plasma. Statistical significance (q<0.05) at stage I is indicated by blue colour. Panel (d) shows trace plots of 5hmC (left) and 5mC (right) fractions in an example region that showed increased 5hmC in stage I plasma and decreased 5mC in stage IV plasma.

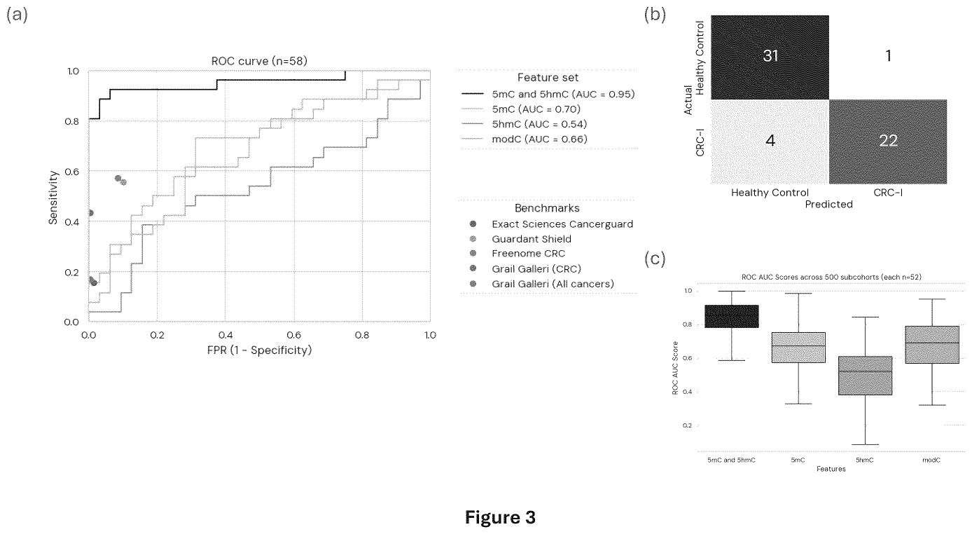

FIG. 3 depicts performance metrics for models using cytosine methylation for predicting colorectal cancer as described herein. Panel (a) shows receiver operating characteristic curves for modC, 5mC, 5hmC, and 5mC and 5hmC models. Panel (b) shows a confusion matrix for predictions from the model using 5mC and 5hmC features with prediction threshold 0.5. Panel (c) shows a box plot showing AUCs across 500 sub-cohorts for modC, 5mC, 5hmC, and 5mC and 5hmC models.

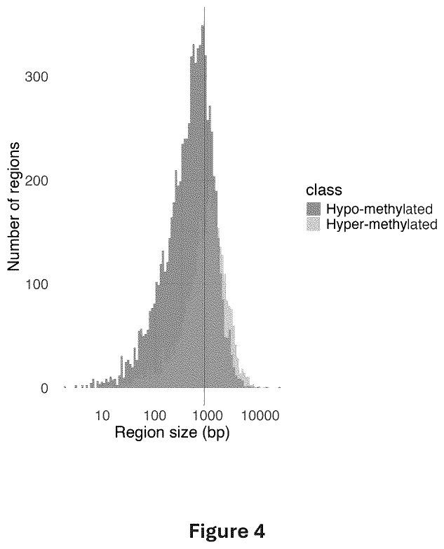

FIG. 4 depicts a histogram showing the size of hypo- and hyper-methylated DMRs from TCGA tissue data as analyzed herein.

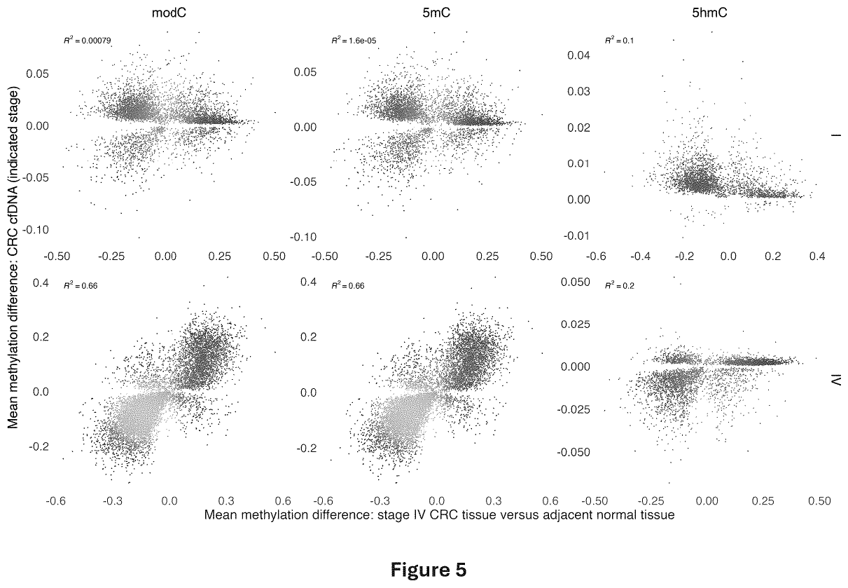

FIG. 5 depicts scatterplots showing mean methylation differences in stage IV CRC tissue (versus adjacent normal tissue) and mean methylation differences in stage I (top) and IV (bottom) cfDNA versus healthy control cfDNA. Each plot is annotated with the coefficient of determination R².

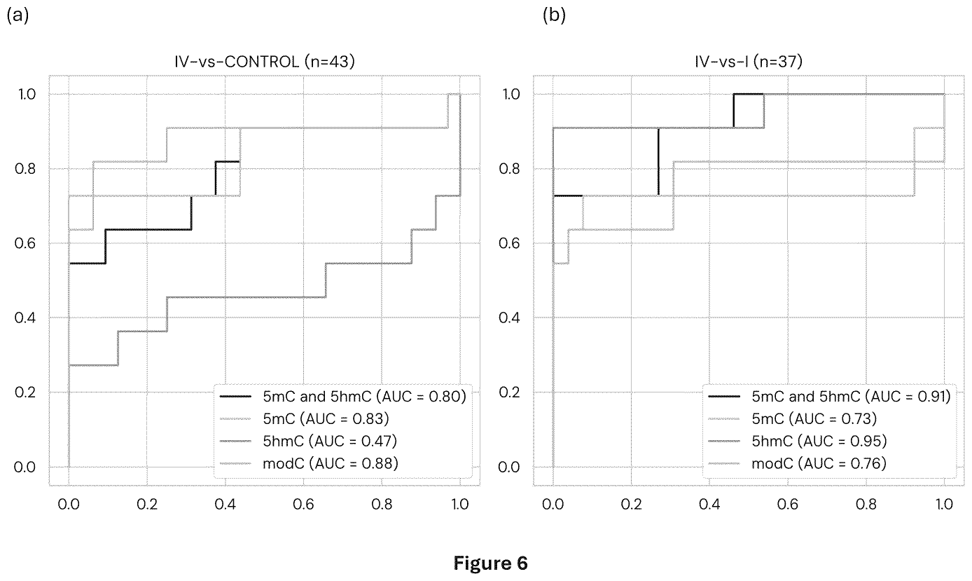

FIG. 6 shows receiver operating characteristic curves for modC, 5mC, 5hmC, and 5mC and 5hmC models for (a) stage IV CRC versus healthy control cfDNA, and (b) stage IV versus stage I CRC cfDNA.

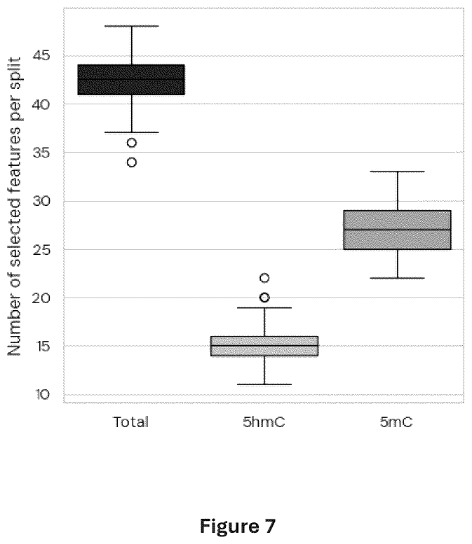

FIG. 7 depicts numbers of features selected by the LASSO classifier when using both 5mC and 5hmC as features (e.g. when both 5mC and 5hmC are analyzed in cfDNA and used as features in the classifier). Each box plot represents the distribution across the 58 cross-validation splits, with the total number of features on the left, 5hmC features in the middle, and 5mC features on the right.

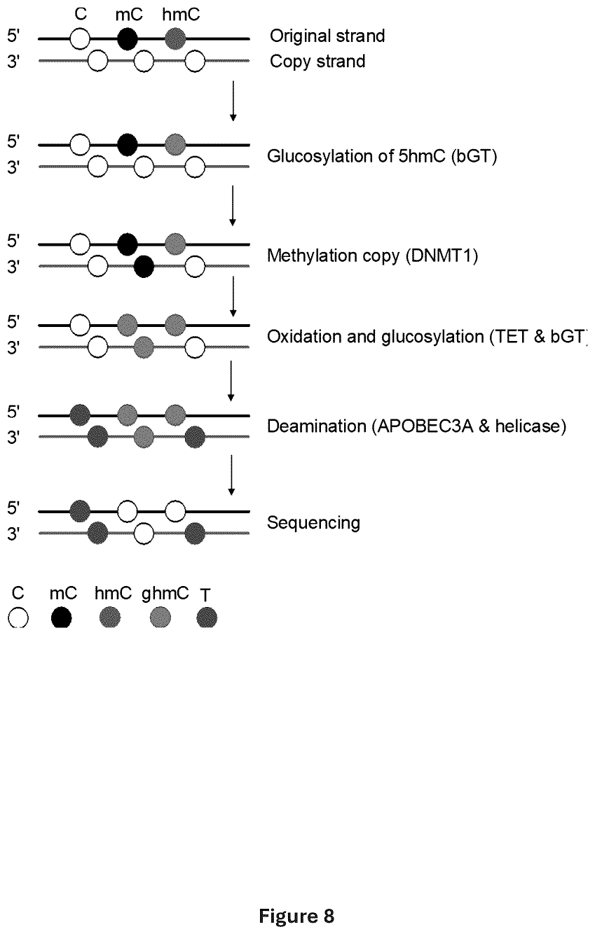

FIG. 8 depicts a method for differentiating and identifying cytosine, mC, and hmC in a DNA sequence, in accordance with embodiments described herein and Examples 1 and 2. In FIG. 8, glycosylation of 5hmC on the first polynucleotide, for example by using BGT and UDPG can be performed to glycosylate 5hmC on the first polynucleotide to yield 5-glucosylhydroxymethylcytosine (₅ghmC or ghmC). Such glycosylation can protect the 5hmC from chemical transformations (e.g. later deamination). In some cases, DNMT1 can be used to methylate, for example cytosine bases, on the second polynucleotide where the methylation is at least in part directed by methylated bases, for example mC, on the first polynucleotide. In some embodiments, uracil can be read as a thymine by a sequencer. Oxidation and glycosylation can then be performed (e.g., using a ten-eleven translocation methylcytosine dioxygenase enzyme (TET) and BGT, respectively) to convert 5mC to 5ghmC. Deamination of cytosine to yield thymine can be performed, for example by using Apolipoprotein B mRNA editing enzyme, catalytic polypeptide-like (APOBEC—such as, for example, APOBEC3A) and a helicase, or fragments thereof, and both strands can be sequenced. The resulting sequencing data can be decoded to determine which bases are cytosine, 5mC, and 5 hmC. In some embodiments, another reagent can be used to yield single stranded DNA in lieu of or in addition to the helicase. Examples of such reagents can comprise, for example, formamide or sodium hydroxide (NaOH). Where the first polynucleotide before processing contains a non-methylated cytosine residue, two thymine residues in physical proximity on the first and second polynucleotides can be read during sequencing, e.g., in a CpG context. Where the first polynucleotide before processing contains a 5mC residue, two cytosine residues in physical proximity on the two strands (e.g., the first and second polynucleotides) can be read during sequencing, e.g., in a CpG context. Where the first polynucleotide before sequencing contains a 5hmC residue, a cytosine (first polynucleotide) and thymine (second polynucleotide) in physical proximity on the two strands (e.g., the first and second polynucleotides) can be read during sequencing, e.g., in a CpG context.

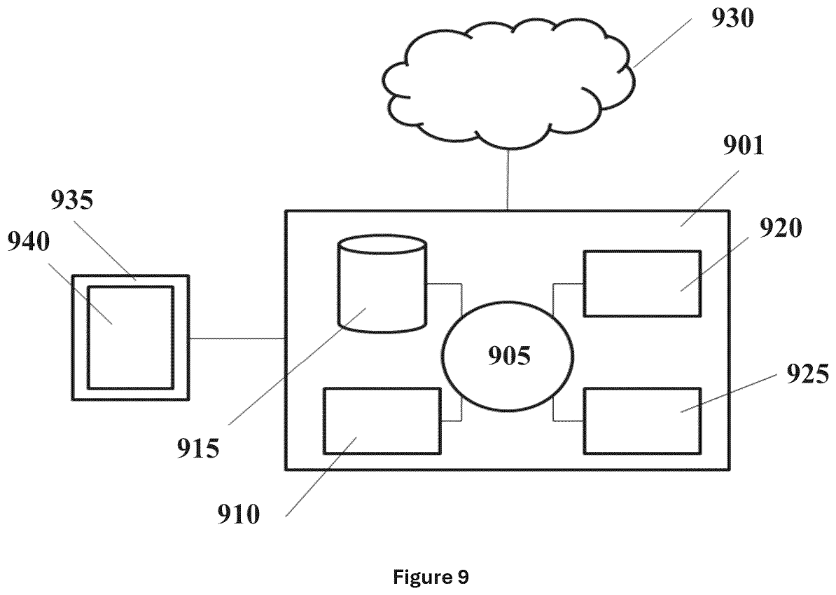

FIG. 9 shows a schematic of a computer system that is programmed or otherwise configured to implement methods of the present disclosure.

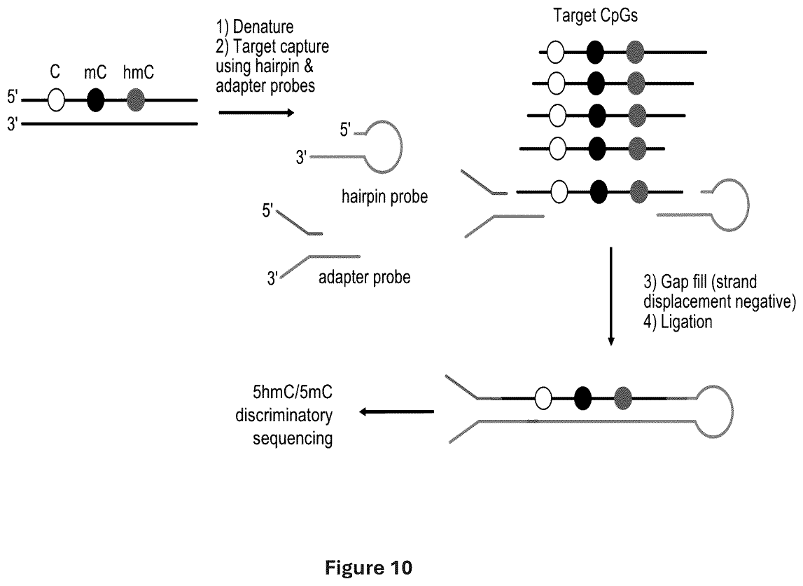

FIG. 10 shows a schematic illustrating targeted capture and subsequent processing of a polynucleotide comprising cytosine (white circle), 5-methylcytosine (black circle), or 5-hydroxymethylcytosine (red circle) that is compatible with 6-letter sequencing analysis as described herein.

### DETAILED DESCRIPTION

The inventors, without wishing to be bound by theory, have recognized that in regions associated with disease that are undergoing differential methylation: (a) amongst the earliest changes are from mC to hmC; and (b) because of the higher prevalence of mC to hmC in healthy subjects the relative magnitude of these changes is indicative of early-stage disease. The inventors have recognized that due to the biological processes underlying DNA methylation these initial changes will likely be seen across indications in regions of the genome where there are differences in methylation state between healthy and patients with advanced stage disease.

Accordingly, described herein are methods that utilize mC, hmC, or combinations thereof to detect disease states and disease stages in subjects suspected of having a disease or having a disease.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

Certain ranges are presented herein with numerical values being preceded by the term “about.” The term “about” is generally used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

In general, terms used herein are generally intended as “open” terms (e.g., the term “including” is to be interpreted as “including but not limited to,” the term “having” is to be interpreted as “having at least,” the term “includes” is to be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases need not be construed to imply that the introduction of a claim recitation by the indefinite articles “a” or “an” limits any particular claim containing such introduced claim recitation to embodiments containing solely one such recitation, even when the same claim includes the introductory phrases “one or more” or “at least one” and indefinite articles such as “a” or “an” (e.g., “a” or “an” is to be interpreted to mean “at least one” or “one or more”); the same holds true for the use of definite articles used to introduce claim recitations. In addition, even if a specific number of an introduced claim recitation is explicitly recited, those skilled in the art will recognize that such recitation is to be interpreted to mean at least the recited number (e.g., the bare recitation of “two recitations,” without other modifiers, generally signifies at least two recitations, or two or more recitations). Furthermore, in those instances where a convention analogous to “at least one of A, B, and C, etc.” is used, in general such a construction is intended in the sense one having skill in the art may understand the convention (e.g., “a system having at least one of A, B, and C” may include but not be limited to systems that have A alone, B alone, C alone, A and B together. A and C together, B and C together, or A, B, and C together, etc.). In those instances where a convention analogous to “at least one of A, B, or C, etc.” is used, in general such a construction is intended in the sense one having skill in the art may understand the convention (e.g., “a system having at least one of A, B, or C” may include but not be limited to systems that have A alone, B alone, C alone, A and B together, A and C together, B and C together, or A, B, and C together, etc.).

In general, as used herein, virtually any disjunctive word and/or phrase presenting two or more alternative terms, is to be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups herein, the disclosure is also generally intended to encompass any individual member or subgroup of members of the Markush group.

In general, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, a range generally includes each individual member. Thus, for example, a group having 1-3 articles refers to groups having 1, 2, or 3 articles. Similarly, a group having 1-5 articles refers to groups having 1, 2, 3, 4, or 5 articles, and so forth.

As used herein, the singular forms “a” “an”, and “the” generally include plural referents unless the context clearly dictates otherwise. For example, the term “a RNA sensor” refers to one or more RNA sensors, e.g., a single RNA sensor and multiple RNA sensors. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Reference to an item in the singular is generally to be understood as including the plural and vice versa unless explicitly stated otherwise or clear from the text. Grammatical conjunctions are intended to express any and all disjunctive conjunctions and conjunctions of conjunctive clauses, sentences, words, and the like, unless otherwise stated or clear from the context. Thus, the term “or” is generally understood to be an inclusive “or” encompassing both alternatives.

The terms “oligonucleotide”. “polynucleotide” and “nucleic acid,” generally used interchangeably herein, generally refer to a polymeric form of nucleotides of any length, either ribonucleotides or deoxynucleotides. Thus, this term includes, but is not limited to, single-, double-, or multi-stranded DNA or RNA, genomic DNA, complementary DNA (cDNA), DNA-RNA hybrids, cell-free nucleic acids (cfNA), cell-free DNA (cfDNA), cell-free RNA (cfRNA), or a polymer including purine and pyrimidine bases or other natural, chemically modified, biochemically modified, non-natural, or derivatized nucleotide bases. The terms “oligonucleotide”, “polynucleotide”, and “nucleic acid” generally are understood to include, as applicable to the embodiment being described, single-stranded (such as sense or antisense) and double-stranded polynucleotides.

The terms “peptide,” “polypeptide.” and “protein” are generally used interchangeably herein, and refer to a polymeric form of amino acids of any length, which can include coded and non-coded amino acids, chemically or biochemically modified or derivatized amino acids, and polypeptides having modified peptide backbones.

The term “cell free nucleic acid” (cfNA) generally refers to nucleic acid fragments that circulate in an individual's body (e.g., bloodstream) and originate from one or more healthy cells and/or from one or more cancerous cells. Additionally, cfNA may come from other sources such as viruses, fetuses, etc. cfNA can include cell-free DNA (cfDNA) or cell-free RNA (cfRNA)

The term “methylation” as used herein generally refers the addition of a methyl group to a DNA molecule (e.g. a base reside of a DNA molecule). For example, a hydrogen atom on the pyrimidine ring of a cytosine base can be converted to a methyl group, forming 5-methylcytosine (5mC). The methyl group can be in an unoxidized state, such as in the case of 5mC, or the methyl group can be in an oxidized state, such as in the case of 5-hydroxymethylcytosine (5hmC), 5-formylcytosine (5fC), or 5-carboxylcytosine (5caC).

The term “CpG sites” generally refers to a region of a DNA molecule which has in linear order from 5′ to 3′ a cytosine nucleotide followed by a guanine nucleotide. “CpG” is generally an abbreviation for 5′-C-phospho-G-3′, e.g., cytosine and guanine are separated by only one phosphate group; the phosphate links any two nucleotides in the DNA together. Cytosine in CpG dinucleotides can be methylated to form 5-mC, 5hmC, 5C, or 5caC. “CpG sites” are generally the most common methylation site, but methylation can occur at other sites. For example, DNA methylation may occur in cytosines at CHG and CHH sites, where H is adenine, cytosine or thymine. Cytosine methylation in the form of 5-hydroxymethylcytosine may also assessed (see e.g., WO 2010/037001 and WO 2011/127136, which are incorporated herein by reference), and features thereof, using the methods and procedures disclosed herein.

The term “fragment” as used herein can generally refer to a fragment of a nucleic acid molecule. For example, in one embodiment, a fragment can refer to a cfDNA molecule in a blood or plasma sample, or a cfDNA molecule that has been extracted from a blood or plasma sample. An amplification product of a cfDNA molecule may also be referred to as a “fragment.” In another embodiment, the term “fragment” refers to a sequencing read, or set of sequencing reads, that have been processed for subsequent analysis (e.g., for in machine-learning based classification), as described herein. For example, raw sequencing reads can be aligned to a reference genome and matching paired end sequence reads assembled into a longer fragment for subsequent analysis.

The term “sequencing,” as used herein generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®)). Alternatively, or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject.

The term “sequencing reads” as used herein generally refers a string of nucleic acid bases corresponding to a sequence of a nucleic acid molecule or fragment that has been sequenced. Sequencing reads can be obtained through various methods provided herein or by other suitable methods.

The term “sequencing depth” as used herein generally refers to the count of the number of times a given target nucleic acid within a sample has been sequenced (e.g., the count of sequence reads at a given target region). Increasing sequencing depth can reduce required amounts of nucleic acids required to assess a disease state (e.g., cancer or cancer tissue of origin).

The term “sample,” as used herein generally refers to a biological sample of a subject. The biological sample may comprise any number of macromolecules, for example, cellular macromolecules. The sample may be a biological particle sample. e.g., a cell or nuclei sample. The sample may be a cell line or cell culture sample. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “subject,” as used herein generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

As used herein the term “chemotherapeutic agent” generally refers to a cytostatic or cytotoxic agent (i.e., a compound) used to eliminate the growth or proliferation of undesirable cells, for example cancer cells. In some cases, “chemotherapeutic agent” generally refers to a cytotoxic or cytostatic agent used to treat a proliferative disorder, for example cancer. The cytotoxic effect of the agent can be, but is not required to be, the result of one or more of nucleic acid intercalation or binding, DNA or RNA alkylation, inhibition of RNA or DNA synthesis, the inhibition of another nucleic acid-related activity (e.g., protein synthesis), or any other cytotoxic effect. A “chemotherapeutic agent” can include, but is not limited to, DNA damaging compounds and other chemicals that can kill cells. “DNA damaging chemotherapeutic agents” can generally include, but are not limited to, alkylating agents, DNA intercalators, protein synthesis inhibitors, inhibitors of DNA or RNA synthesis, DNA base analogs, topoisomerase inhibitors, and telomerase inhibitors or telomeric DNA binding compounds. For example, alkylating agents include alkyl sulfonates, such as busulfan, improsulfan, and piposulfan; aziridines, such as a benzodizepa, carboquone, meturedepa, and uredepa; ethylenimines and methylmelamines, such as altretamine, triethylenemelamine, triethylenephosphoramide, triethylenethiophosphoramide, and trimethylolmelamine; nitrogen mustards such as chlorambucil, chlomaphazine, cyclophosphamide, estramustine, iphosphamide, mechlorethamine, mechlorethamine oxide hydrochloride, melphalan, novembichine, phenesterine, prednimustine, trofosfamide, and uracil mustard; and nitroso ureas, such as carmustine, chlorozotocin, fotemustine, lomustine, nimustine, and ranimustine. “Chemotherapeutic agents” can also include antibiotics such as dactinomycin, daunorubicin, doxorubicin, idarubicin, bleomycin sulfate, mytomycin, plicamycin, and streptozocin. Chemotherapeutic antimetabolites include mercaptopurine, thioguanine, cladribine, fludarabine phosphate, fluorouracil (5-FU), floxuridine, cytarabine, pentostatin, methotrexate, and azathioprine, acyclovir, adenine β-1-D-arabinoside, amethopterin, aminopterin, 2-aminopurine, aphidicolin, 8-azaguanine, azaserine, 6-azauracil, 2′-azido-2′-deoxynucleosides, 5-bromodeoxycyti dine, cytosine β-1-D-arabinoside, diazooxynorleucine, dideoxynucleosides, 5-fluorodeoxycytidine, 5-fluorodeoxyuridine, and hydroxyurea. “Chemotherapeutic agents” can also include protein synthesis inhibitors such as abrin, aurintricarboxylic acid, chloramphenicol, colicin E3, cycloheximide, diphtheria toxin, edeine A, emetine, erythromycin, ethionine, fluoride, 5-fluorotryptophan, fusidic acid, guanylyl methylene diphosphonate and guanylyl imidodiphosphate, kanamycin, kasugamycin, kirromycin, and O-methyl threonine. Additional protein synthesis inhibitors include modeccin, neomycin, norvaline, pactamycin, paromomycine, puromycin, ricin, shiga toxin, showdomycin, sparsomycin, spectinomycin, streptomycin, tetracycline, thiostrepton, and trimethoprim. Inhibitors of DNA synthesis, include alkylating agents such as dimethyl sulfate, mitomycin C, nitrogen and sulfur mustards; intercalating agents, such as acridine dyes, actinomycins, adriamycin, anthracenes, benzopyrene, ethidium bromide, propidium diiodide-intertwining; and other agents, such as distamycin and netropsin. Topoisomerase inhibitors, such as coumermycin, nalidixic acid, novobiocin, and oxolinic acid; inhibitors of cell division, including colcemide, colchicine, vinblastine, and vincristine; and RNA synthesis inhibitors including actinomycin D, α-amanitine and other fungal amatoxins, cordycepin (3′-deoxyadenosine), dichlororibofuranosyl benzimidazole, rifampicine, streptovaricin, and streptolydigin also can be used as the DNA damaging compound.

Percent identity between or within polypeptide sequences described herein can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

Included in the current disclosure are variants of any of the polypeptides described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally or alternatively, by comparing aligned sequences of homologous proteins from different species, conservative substitutions can be identified by locating amino acid residues that have been mutated between species (e.g., non-conserved residues) without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%, or 100% sequence identity to any one of the polypeptide protein sequences described herein.

In some embodiments, such conservatively substituted variants are functional variants. Such functional variants can encompass sequences with substitutions such that the activity of critical active site residues of the endonuclease are not disrupted. In some embodiments, a functional variant of any of the proteins described herein lacks substitution of at least one conserved or functional residue.

Conservative substitution tables providing functionally similar amino acids are available from a variety of references (see, for e.g., Creighton. Proteins: Structures and Molecular Properties (W H Freeman & Co.; 2nd edition (December 1993), which is incorporated by reference in its entirety herein). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

In some aspects, the present disclosure provides for a polypeptide or use of a polypeptide comprising or encoding any of the sequences described in Table A, or a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about %%, at least about 97%, at least about 98%, or at least about 99%, or 100% sequence identity to any of the sequences described in Table A.

TABLE A
Sequences of Genes and Components Described Herein

| SEQ ID | | |
|---|---|---|
| NO: | DESCRIPTION | SEQUENCE |
| 1 | α- | MKIAIINMGNNVINFKTVPSSETIYLFKVISEMGLNVDIISL |
| | glucosyl- | KNGVYTKSFDEVDVNDYDRLIVVNSSINFFGGKPNLAILS |
| | transferase | AQKFMAKYKSKIYYLFTDIRLPFSQSWPNVKNRPWAYLY |
| | (AGT) | TEEELLIKSPIKVISQGINLDIAKAAHKKVDNVIEFEYFPIE |
| | (RCSB | QYKIHMNDFQLSKPTKKTLDVIYGGSFRSGQRESKMVEF |
| | Accession | LFDTGLNIEFFGNAREKQFKNPKYPWTKAPVFTGKIPMN |
| | No. 1J39, | MVSEKNSQAIAALIIGDKNYNDNFITLRVWETMASDAVM |
| | UniProtKB | LIDEEFDTKHRIINDARFYVNNRAELIDRVNELKHSDVLR |
| | Accession | KEMLSIQHDILNKTRAKKAEWQDAFKKAIDL |
| | No. | |
| | P04547) | |
| 2 | β- | MRICIFMARGLEGCGVTKFSLEQRDWFIKNGHEVTLVYA |
| | glucosyl- | KDKSFTRTSSHDHKSFSIPVILAKEYDKALKLVNDCDILII |
| | transferase | NSVPATSVQEATINNYKKLLDNIKPSIRVVVYQHDHSVLS |
| | (BGT) | LRRNLGLEETVRRADVIFSHSDNGDFNKVLMKEWYPETV |
| | (RCSB | SLFDDIEEAPTVYNFQPPMDIVKVRSTYWKDVSEINMNIN |
| | Accession | RWIGRTTTWKGFYQMFDFHEKFLKPAGKSTVMEGLERSP |
| | No. 1YA6, | AFIAIKEKGIPYEYYGNREIDKMNLAPNQPAQILDCYINSE |
| | UniprotKB | MLERMSKSGFGYQLSKLNQKYLQRSLEYTHLELGACGTI |
| | Accession. | PVFWKSTGENLKFRVDNTPLTSHDSGIIWFDENDMESTFE |
| | No. | RIKELSSDRALYDREREKAYEFLYQHQDSSFCFKEQFDIIT |
| | P04519) | K |
| 3 | DNA | MPARTAPARVPTLAVPAISLPDDVRRRLKDLERDSLTEKE |
| | (cytosine- | CVKEKLNLLHEFLQTEIKNQLCDLETKLRKEELSEEGYLA |
| | 5)- | KVKSLLNKDLSLENGAHAYNREVNGRLENGNQARSEAR |
| | methyl- | RVGMADANSPPKPLSKPRTPRRSKSDGEAKPEPSPSPRITR |
| | transferase 1 | KSTRQTTITSHFAKGPAKRKPQEESERAKSDESIKEEDKD |
| | (DNMT1) | QDEKRRRVTSRERVARPLPAEEPERAKSGTRTEKEEERDE |
| | human | KEEKRLRSQTKEPTPKQKLKEEPDREARAGVQADEDEDG |
| | (UniprotKB | DEKDEKKHRSQPKDLAAKRRPEEKEPEKVNPQISDEKDE |
| | Accession | DEKEEKRRKTTPKEPTEKKMARAKTVMNSKTHPPKCIQC |
| | No. | GQYLDDPDLKYGQHPPDAVDEPQMLTNEKLSIFDANESG |
| | P26358) | FESYEALPQHKLTCFSVYCKHGHLCPIDTGLIEKNIELFFS |
| | | GSAKPIYDDDPSLEGGVNGKNLGPINEWWITGFDGGEKA |
| | | LIGFSTSFAEYILMDPSPEYAPIFGLMQEKIYISKIVVEFLQS |
| | | NSDSTYEDLINKIETTVPPSGLNLNRFTEDSLLRHAQFVVE |
| | | QVESYDEAGDSDEQPIFLTPCMRDLIKLAGVTLGQRRAQ |
| | | ARRQTIRHSTREKDRGPTKATTTKLVYQIFDTFFAEQIEK |
| | | DDREDKENAFKRRRCGVCEVCQQPECGKCKACKDMVK |
| | | FGGSGRSKQACQERRCPNMAMKEADDDEEVDDNIPEMP |
| | | SPKKMHQGKKKKQNKNRISWVGEAVKTDGKKSYYKKV |
| | | CIDAETLEVGDCVSVIPDDSSKPLYLARVTALWEDSSNGQ |
| | | MFHAHWFCAGTDTVLGATSDPLELFLVDECEDMQLSYIH |
| | | SKVKVIYKAPSENWAMEGGMDPESLLEGDDGKTYFYQL |
| | | WYDQDYARFESPPKTQPTEDNKFKFCVSCARLAEMRQK |
| | | EIPRVLEQLEDLDSRVLYYSATKNGILYRVGDGVYLPPEA |
| | | FTFNIKLSSPVKRPRKEPVDEDLYPEHYRKYSDYIKGSNL |
| | | DAPEPYRIGRIKEIFCPKKSNGRPNETDIKIRVNKFYRPENT |
| | | HKSTPASYHADINLLYWSDEEAVVDFKAVQGRCTVEYG |
| | | EDLPECVQVYSMGGPNRFYFLEAYNAKSKSFEDPPNHAR |
| | | SPGNKGKGKGKGKGKPKSQACEPSEPEIEIKLPKLRTLDV |
| | | FSGCGGLSEGFHQAGISDTLWAIEMWDPAAQAFRLNNPG |
| | | STVFTEDCNILLKLVMAGETTNSRGQRLPQKGDVEMLCG |
| | | GPPCQGFSGMNRFNSRTYSKFKNSLVVSFLSYCDYYRPRF |
| | | FLLENVRNFVSFKRSMVLKLTLRCLVRMGYQCTFGVLQA |
| | | GQYGVAQTRRRAIILAAAPGEKLPLFPEPLHVFAPRACQL |
| | | SVVVDDKKFVSNITRLSSGPFRTITVRDTMSDLPEVRNGA |
| | | SALEISYNGEPQSWFQRQLRGAQYQPILRDHICKDMSALV |
| | | AARMRHIPLAPGSDWRDLPNIEVRLSDGTMARKLRYTHH |
| | | DRKNGRSSSGALRGVCSCVEAGKACDPAARQFNTLIPWC |
| | | LPHTGNRHNHWAGLYGRLEWDGFFSTTVTNPEPMGKQG |
| | | RVLHPEQHRVVSVRECARSQGFPDTYRLFGNILDKHRQV |
| | | GNAVPPPLAKAIGLEIKLCMLAKARESASAKIKEEEAAKD |
| 4 | DNA | MTTALTFGGGLFKDNTKFDIDMRGTADGAVNGGNIPNSQ |
| | (cytosine- | SQKRKRASPSPEIESEEDGDDWYEIDYIADSRVIRRKGRQI |
| | 5)- | LQYLIHWAGYAVHERTWEDEDGIGGEDCALVQEFYRKN |
| | methyl- | PGKPRLSPSSVRKEVKLARMVEVVITTRRIDGKSRAASST |
| | transferase 5 | DQPSPHRLGITSPQANNIGGEDPNPSLTRRPVRSTVSEIAK |
| | (DNMT5) | RPTSKKVHPNKKCKASSDDESDFVFEEGEWDEDEDDDN |
| | Cryptococcus | DVDFRSSEDDEDDEQERSAEEPESDEEIIKPAKKTKSSLPK |
| | neoformans | AKLRPKPANLGGFVTGVRPLNQGLDIKAAVRNMSDDLPP |
| | (UniprotKB | ISDIEAMFDHLVSRIPDIVELVRQLNGRKLRVATMCSGTE |
| | Accession | SPLLALNMIAKAIKAQHGLTLAFEHVFSCEIEPFKQAYIER |
| | No. | NFTPPILFRDVTELGKKRAHTAYGSMVDVPGDVDILIAGT |
| | J9VI03) | SCVDYSNLNNVQQDIDANGESGRTFRGMLQWVKKHQPP |
| | | IVILENVCNAPWDKVVEYFGQIDYDAQYTRLDTKEFYIPH |
| | | TRTRVYLFATPSSSESDDLPEKWAQTVKDLRRPWSSPFEA |
| | | FLLHTDDPNIHRARLELASARAQTDGTSRKTTDWNRCES |
| | | RHQRARQDEALGLLRPLTSWQEAGVCKGLDWTWNDWL |
| | | LAQTERVVDLLEISTLRMAKDGIDSGFKACIWNVSQNVD |
| | | RQTGSSKTALAPCLTPNMIPWVTIRGGPVTGREALALQGI |
| | | PVRELLLTSENEDQLADLAGNAMTTTVVGSAMIAALKV |
| | | ACHKITEGANPEKEAALILEKEAVDDEQVANRIIGEDYLE |
| | | HHDLDLAKVTKSNLSEILDLACRSSRHCQCEGQSGTAPNI |
| | | LECQECSYRACKSCGGRPEHVYAPCANQRVEPAEFEKRF |
| | | KGLLPMRVRIAGLTDQCLNAVRKAAEKSNKGSVNDNDW |
| | | QLWSTALLEGIHDAEFRFRYLKRQSTWTAVYEARRAMLS |
| | | LVLRNQIPEWRLTIKAPASEPNNSQLRALLLHPVARLQIDI |
| | | AGQDVLCGPWELCIPSMKTIDIEITGKGELLPSWQASLGL |
| | | QGPFANTTRWSEVEISLQAEDENTLDRKLSGTYQLLPRCG |
| | | QAMSSLHKKRPDLSDDGLPQLYFFLDPTRCGESREDRYV |
| | | FSTSTERLDYGTERPVIARLDSHWREGNEKQRKVKLDVS |
| | | GAWVKCPEAHLTAIGGDDIAVVANDAAANEIHRDRATF |
| | | AIPSSASAISASLTTEGCSHAMALLSCRVPLDPTHSESMW |
| | | RRGAWAEIDLSHQGNTTFANLAWITERLPPLDGLKNWA |
| | | HIADDVSEHVCERCAPRPPKIHWIKREGKANKKGNKTKS |
| | | TIIAFEDKLEAGQYEHALKHRPSPFVVQLRLDQDIGSFRIG |
| | | LNIVSLAHRALSRLPPTTSEHKISLSWRLTPGHVTESPQPR |
| | | RVFILPSNKQDPENSQPEAFKLPLRKEQLRSLWWMLEQE |
| | | KATGKTHTFVEEEISESLLPAVGWRAEGKAERPVMVRGG |
| | | VIADQVGYGKTVISIALVAQTLSLPAPEPATPGLIDLKATL |
| | | IVVPGHLSKQWPNEIARFTGSMFKVIVIQGMKDLQEKTIA |
| | | ELGKADIIVMASEIFESDVYWSRLEYLSAQPREWLHDTQ |
| | | GGRFFCDRLDAAMESLVSQTKILKEKGSEAAMRAMEDK |
| | | KKSLVDNVGSKKEVHTAVNFGKRMKGQAYRDKHDSDS |
| | | KAKPITKEELERWEASEDEDDDENSKTYIPIPKFHSFTGSE |
| | | SIFSASVKKDYKLLPNPVLHMFRFRRVIADEFTYLQKKSL |
| | | AAVLRLSSSYRWILSGTPPVSDFAAIRSIATFMGIHLGVED |
| | | DGEGDVQYQKARAKDQTQAEKFHAFREVHSRAWHNRR |
| | | DELAQEFLNVFVRQNIAEIEDIPTVEHIHTFKLPASEGAVY |
| | | LELEHHLQALEMQARKETKFKNVSQGDRNARLEEALSDS |
| | | KTAEEALLKRCCHFTLDLSDKTQDAKSAQEACDHITSAR |
| | | ARQLLACQEDLSRSVNQAIALHGWIKKKGGFSKNDDERQ |
| | | PFAEWIAFSSNISKHQGDIEAARILLKVIEKCGVKDGNIPPS |
| | | PSDKQSPSIASGAKMDDVKWQLREQTHLLRKLVKELVA |
| | | RVRSLRFFEVVRKIQKGKSDAQIVLESSECGHKPSTNPDIE |
| | | MAILSCCGHVACHKCMRKAAASQRCVKSGECQAAVRPT |
| | | NIVKVSSLGVEGELSSGRYGAKLEHLVNLIHSIPKNERVL |
| | | VFLQWEDLAGKVSEALSAGRIPHVTLSGSAKSRANTLDR |
| | | FQSTNADSARVLLLKMNDASAAGSNLTTANHAVFLGPLF |
| | | TNSLFNYRAVETQAIGRVRRYGQQKKVHIHRLLALDTID |
| | | MTIFNARRTELKEKTDWEEIPQEEYKGRGSSISMTNEKRT |
| | | PTLTVKSNPFKRSSSWALASSFRSKKRSMEARDAEGVSD |
| | | DDENSELSDII |
| 5 | apolipoprotein | MEASPASGPRHLMDPHIFTSNFNNGIGRHKTYLCYEVERL |
| | B mRNA | DNGTSVKMDQHRGFLHNQAKNLLCGFYGRHAELRFLDL |
| | editing | VPSLQLDPAQIYRVTWFISWSPCFSWGCAGEVRAFLQEN |
| | enzyme, | THVRLRIFAARIYDYDPLYKEALQMLRDAGAQVSIMTYD |
| | catalytic | EFKHCWDTFVDHQGCPFQPWDGLDEHSQALSGRLRAILQ |
| | polypeptide- | NQGN |
| | like 3A | |
| | (APOBEC3A) | |
| | human | |
| | (UniprotKB | |
| | Accession | |
| | No. | |
| | P31941) | |
| 6 | UvrD | MDVSYLLDSLNDKQREAVAAPRSNLLVLAGAGSGKTRV |
| | helicase, E. | LVHRIAWLMSVENCSPYSIMAVTFTNKAAAEMRHRIGQL |
| | coli | MGTSQGGMWVGTFHGLAHRLLRAHHMDANLPQDFQIL |
| | (UniprotKB | DSEDQLRLLKRLIKAMNLDEKQWPPRQAMWYINSQKDE |
| | Accession | GLRPHHIQSYGNPVEQTWQKVYQAYQEACDRAGLVDFA |
| | No. | ELLLRAHELWLNKPHILQHYRERFTNILVDEFQDTNNIQY |
| | P03018) | AWIRLLAGDTGKVMIVGDDDQSIYGWRGAQVENIQRFL |
| | | NDFPGAETIRLEQNYRSTSNILSAANALIENNNGRLGKKL |
| | | WTDGADGEPISLYCAFNELDEARFVVNRIKTWQDNGGA |
| | | LAECAILYRSNAQSRVLEEALLQASMPYRIYGGMRFFER |
| | | QEIKDALSYLRLIANRNDDAAFERVVNTPTRGIGDRTLDV |
| | | VRQTSRDRQLTLWQACRELLQEKALAGRAASALQRFME |
| | | LIDALAQETADMPLHVQTDRVIKDSGLRTMYEQEKGEKG |
| | | QTRIENLEELVTATRQFSYNEEDEDLMPLQAFLSHAALEA |
| | | GEGQADTWQDAVQLMTLHSAKGLEFPQVFIVGMEEGMF |
| | | PSQMSLDEGGRLEEERRLAYVGVTRAMQKLTLTYAETR |
| | | RLYGKEVYHRPSRFIGELPEECVEEVRLRATVSRPVSHQR |
| | | MGTPMVENDSGYKLGQRVRHAKFGEGTIVNMEGSGEHS |
| | | RLQVAFQGQGIKWLVAAYARLESV |
| 7 | PcrA | MNFLSEQLLAHLNKEQQEAVRTTEGPLLIMAGAGSGKTR |
| | helicase, | VLTHRIAYLMAEKHVAPWNILAITFTNKAAREMRERVQS |
| | Geobacillus | LLGGAAEDVWISTFHSMCVRILRRDIDRIGINRNFSILDPT |
| | stearo- | DQLSVMKTILKEKNIDPKKFEPRTILGTISAAKNELLPPEQ |
| | thermophilus | FAKRASTYYEKVVSDVYQEYQQRLLRNHSLDFDDLIMTT |
| | (UniprotKB | IQLFDRVPDVLHYYQYKFQYIHIDEYQDTNRAQYTLVKK |
| | Accession | LAERFQNICAVGDADQSTYRWRGADIQNILSFERDYPNAK |
| | No. | VILLEQNYRSTKRILQAANEVIEHNVNRKPKRIWTENPEG |
| | P56255) | KPILYYEAMNEADEAQFVAGRIREAVERGERRYRDFAVL |
| | | YRTNAQSRVMEEMLLKANIPYQIVGGLKFYDRKEIKDIL |
| | | AYLRVIANPDDDLSLLRIINVPKRGIGASTIDKLVRYAAD |
| | | HELSLFEALGELEMIGLGAKAAGALAAFRSQLEQWTQLQ |
| | | EYVSVTELVEEVLDKSGYREMLKAERTIEAQSRLENLDEF |
| | | LSVTKHFENVSDDKSLIAFLTDLALISDLDELDGTEQAAE |
| | | GDAVMLMTLHAAKGLEFPVVFLIGMEEGIFPHNRSLEDD |
| | | DEMEEERRLAYVGITRAEEELVLTSAQMRTLFGNIQMDP |
| | | PSRFLNEIPAHLLETASRRQAGASRPAVSRPQASGAVGSW |
| | | KVGDRANHRKWGIGTVVSVRGGGDDQELDIAFPSPIGIK |
| | | RLLAKFAPIEKV |
| 8 | TET2 | MEQDRTNHVEGNRLSPFLIPSPPICQTEPLATKLQNGSPLP |
| | human | ERAHPEVNGDTKWHSFKSYYGIPCMKGSQNSRVSPDFTQ |
| | (UniprotKB | ESRGYSKCLQNGGIKRTVSEPSLSGLLQIKKLKQDQKANG |
| | Accession | ERRNFGVSQERNPGESSQPNVSDLSDKKESVSSVAQENA |
| | No. | VKDFTSFSTHNCSGPENPELQILNEQEGKSANYHDKNIVL |
| | Q6N021) | LKNKAVLMPNGATVSASSVEHTHGELLEKTLSQYYPDCV |
| | | SIAVQKTTSHINAINSQATNELSCEITHPSHTSGQINSAQTS |
| | | NSELPPKPAAVVSEACDADDADNASKLAAMLNTCSFQKP |
| | | EQLQQQKSVFEICPSPAENNIQGTTKLASGEEFCSGSSSNL |
| | | QAPGGSSERYLKQNEMNGAYFKQSSVFTKDSFSATTTPPP |
| | | PSQLLLSPPPPLPQVPQLPSEGKSTLNGGVLEEHHHYPNQS |
| | | NTTLLREVKIEGKPEAPPSQSPNPSTHVCSPSPMLSERPQN |
| | | NCVNRNDIQTAGTMTVPLCSEKTRPMSEHLKHNPPIFGSS |
| | | GELQDNCQQLMRNKEQEILKGRDKEQTRDLVPPTQHYL |
| | | KPGWIELKAPRFHQAESHLKRNEASLPSILQYQPNLSNQM |
| | | TSKQYTGNSNMPGGLPRQAYTQKTTQLEHKSQMYQVEM |
| | | NQGQSQGTVDQHLQFQKPSHQVHFSKTDHLPKAHVQSL |
| | | CGTRFHFQQRADSQTEKLMSPVLKQHLNQQASETEPFSN |
| | | SHLLQHKPHKQAAQTQPSQSSHLPQNQQQQQKLQIKNKE |
| | | EILQTFPHPQSNNDQQREGSFFGQTKVEECFHGENQYSKS |
| | | SEFETHNVQMGLEEVQNINRRNSPYSQTMKSSACKIQVS |
| | | CSNNTHLVSENKEQTTHPELFAGNKTQNLHHMQYFPNN |
| | | VIPKQDLLHRCFQEQEQKSQQASVLQGYKNRNQDMSGQ |
| | | QAAQLAQQRYLIHNHANVFPVPDQGGSHTQTPPQKDTQ |
| | | KHAALRWHLLQKQEQQQTQQPQTESCHSQMHRPIKVEP |
| | | GCKPHACMHTAPPENKTWKKVTKQENPPASCDNVQQKS |
| | | HIETMEQHLKQFHAKSLFDHKALTLKSQKQVKVEMSGPV |
| | | TVLTRQTTAAELDSHTPALEQQTTSSEKTPTKRTAASVLN |
| | | NFIESPSKLLDTPIKNLLDTPVKTQYDFPSCRCVEQIIEKDE |
| | | GPFYTHLGAGPNVAAIREIMEERFGQKGKAIRIERVIYTG |
| | | KEGKSSQGCPIAKWVVRRSSSEEKLLCLVRERAGHTCEA |
| | | AVIVILILVWEGIPLSLADKLYSELTETLRKYGTLTNRRCA |
| | | LNEERTCACQGLDPETCGASFSFGCSWSMYYNGCKFARS |
| | | KIPRKFKLLGDDPKEEEKLESHLQNLSTLMAPTYKKLAPD |
| | | AYNNQIEYEHRAPECRLGLKEGRPFSGVTACLDFCAHAH |
| | | RDLHNMQNGSTLVCTLTREDNREFGGKPEDEQLHVLPLY |
| | | KVSDVDEFGSVEAQEEKKRSGAIQVLSSFRRKVRMLAEP |
| | | VKTCRQRKLEAKKAAAEKLSSLENSSNKNEKEKSAPSRT |
| | | KQTENASQAKQLAELLRLSGPVMQQSQQPQPLQKQPPQP |
| | | QQQQRPQQQQPHHPQTESVNSYSASGSTNPYMRRPNPVS |
| | | PYPNSSHTSDIYGSTSPMNFYSTSSQAAGSYLNSSNPMNP |
| | | YPGLLNQNTQYPSYQCNGNLSVDNCSPYLGSYSPQSQPM |
| | | DLYRYPSQDPLSKLSLPPIHTLYQPRFGNSQSFTSKYLGYG |
| | | NQNMQGDGFSSCTIRPNVHHVGKLPPYPTHEMDGHFMG |
| | | ATSRLPPNLSNPNMDYKNGEHHSPSHIIHNYSAAPGMENS |
| | | SLHALHLQNKENDMLSHTANGLSKMLPALNHDRTACVQ |
| | | GGLHKLSDANGQEKQPLALVQGVASGAEDNDEVWSDSE |
| | | QSFLDPDIGGVAVAPTHGSILIECAKRELHATTPLKNPNR |
| | | NHPTRISLVFYQHKSMNEPKHGLALWEAKMAEKAREKE |
| | | EECEKYGPDYVPQKSHGKKVKREPAEPHETSEPTYLRFIK |
| | | SLAERTMSVTTDSTVTTSPYAFTRVTGPYNRYI |
| 9 | TET 2 | MEQDRTTHAEGTRLSPFLIAPPSPISHTEPLAVKLQNGSPL |
| | mouse | AERPHPEVNGDTKWQSSQSCYGISHMKGSQSSHESPHED |
| | (UniprotKB | RGYSRCLQNGGIKRTVSEPSLSGLHPNKILKLDQKAKGES |
| | Accession | NIFEESQERNHGKSSRQPNVSGLSDNGEPVTSTTQESSGA |
| | No. | DAFPTRNYNGVEIQVLNEQEGEKGRSVTLLKNKIVLMPN |
| | Q4JK59) | GATVSAHSEENTRGELLEKTQCYPDCVSIAVQSTASHVN |
| | | TPSSQAAIELSHEIPQPSLTSAQINFSQTSSLQLPPEPAAMV |
| | | TKACDADNASKPAIVPGTCPFQKAEHQQKSALDIGPSRAE |
| | | NKTIQGSMELFAEEYYPSSDRNLQASHGSSEQYSKQKET |
| | | NGAYFRQSSKFPKDSISPTTVTPPSQSLLAPRLVLQPPLEG |
| | | KGALNDVALEEHHDYPNRSNRTLLREGKIDHQPKTSSSQ |
| | | SLNPSVHTPNPPLMLPEQHQNDCGSPSPEKSRKMSEYLM |
| | | YYLPNHGHSGGLQEHSQYLMGHREQEIPKDANGKQTQG |
| | | SVQAAPGWIELKAPNLHEALHQTKRKDISLHSVLHSQTG |
| | | PVNQMSSKQSTGNVNMPGGFQRLPYLQKTAQPEQKAQM |
| | | YQVQVNQGPSPGMGDQHLQFQKALYQECIPRTDPSSEAH |
| | | PQAPSVPQYHFQQRVNPSSDKHLSQQATETQRLSGFLQH |
| | | TPQTQASQTPASQNSNFPQICQQQQQQQLQRKNKEQMPQ |
| | | TFSHLQGSNDKQREGSCFGQIKVEESFCVGNQYSKSSNFQ |
| | | THNNTQGGLEQVQNINKNFPYSKILTPNSSNLQILPSNDT |
| | | HPACEREQALHPVGSKTSNLQNMQYFPNNVTPNQDVHR |
| | | CFQEQAQKPQQASSLQGLKDRSQGESPAPPAEAAQQRYL |
| | | VHNEAKALPVPEQGGSQTQTPPQKDTQKHAALRWLLLQ |
| | | KQEQQQTQQSQPGHNQMLRPIKTEPVSKPSSYRYPLSPPQ |
| | | ENMSSRIKQEISSPSRDNGQPKSIIETMEQHLKQFQLKSLC |
| | | DYKALTLKSQKHVKVPTDIQAAESENHARAAEPQATKST |
| | | DCSVLDDVSESDTPGEQSQNGKCEGCNPDKDEAPYYTHL |
| | | GAGPDVAAIRTLMEERYGEKGKAIRIEKVIYTGKEGKSSQ |
| | | GCPIAKWVYRRSSEEEKLLCLVRVRPNHTCETAVMVIAI |
| | | MLWDGIPKLLASELYSELTDILGKCGICTNRRCSQNETRN |
| | | CCCQGENPETCGASFSFGCSWSMYYNGCKFARSKKPRKF |
| | | RLHGAEPKEEERLGSHLQNLATVIAPIYKKLAPDAYNNQ |
| | | VEFEHQAPDCCLGLKEGRPFSGVTACLDFSAHSHRDQQN |
| | | MPNGSTVVVTLNREDNREVGAKPEDEQFHVLPMYIIAPE |
| | | DEFGSTEGQEKKIRMGSIEVLQSFRRRRVIRIGELPKSCKK |
| | | KAEPKKAKTKKAARKRSSLENCSSRTEKGKSSSHTKLME |
| | | NASHMKQMTAQPQLSGPVIRQPPTLQRHLQQGQRPQQPQ |
| | | PPQPQPQTTPQPQPQPQHIMPGNSQSVGSHCSGSTSVYTR |
| | | QPTPHSPYPSSAHTSDIYGDTNHVNFYPTSSHASGSYLNPS |
| | | NYMNPYLGLLNQNNQYAPFPYNGSVPVDNGSPFLGSYSP |
| | | QAQSRDLHRYPNQDHLTNQNLPPIHTLHQQTFGDSPSKY |
| | | LSYGNQNMQRDAFTTNSTLKPNVHHLATFSPYPTPKMDS |
| | | HFMGAASRSPYSHPHTDYKTSEHHLPSHTIYSYTAAASGS |
| | | SSSHAFHNKENDNIANGLSRVLPGFNHDRTASAQELLYSL |
| | | TGSSQEKQPEVSGQDAAAVQEIEYWSDSEHNFQDPCIGG |
| | | VAIAPTHGSILIECAKCEVHATTKVNDPDRNHPTRISLVLY |
| | | RHKNLFLPKHCLALWEAKMAEKARKEEECGKNGSDHVS |
| | | QKNHGKQEKREPTGPQEPSYLRFIQSLAENTGSVTTDSTV |
| | | TTSPYAFTQVTGPYNTFV |

The methods described herein include using a trained classifier or algorithm to analyze sample data, particularly to performing a classification between a subject having a disease and a subject not having the disease (e.g. a binary classification), or between a subject having an early stage of a disease versus a late stage of the disease.

In supervised learning approaches involving trained classifiers or algorithms, a group of samples from two or more groups are analyzed with a statistical classification method. Differential gene or nucleic acid level data can be discovered that can be used to build a classifier that differentiates between the two or more groups. A new sample can then be analyzed so that the classifier can associate the new sample with one of the two or more groups. Commonly used supervised classifiers include without limitation the neural network (multi-layer perceptron), support vector machines, k-nearest neighbors. Gaussian mixture model, Gaussian, naive Bayes, decision tree and radial basis function (RBF) classifiers. Linear classification methods include Fisher's linear discriminant, LDA, logistic regression, naive Bayes classifier, perceptron, and support vector machines (SVMs). Other classifiers for use with the invention include quadratic classifiers, k-nearest neighbor, boosting, decision trees, random forests, neural networks, pattern recognition. Elastic Net, Golub Classifier, Parzen-window, Iterative RELIEF, Classification Tree. Maximum Likelihood Classifier, Nearest Centroid, Prediction Analysis of Microarrays (PAM), Fuzzy C-Means Clustering, Bayesian networks and Hidden Markov models.

In order to solve a given problem of supervised learning one can consider various operations:
1. Gather a training set. These can include, for example, samples that are from subjects suspected having an autoimmune condition. The training samples are used to “train” the classifier.
2. Determine the input “feature” representation of the learned function. The accuracy of the learned function depends on how the input object is represented. The input object is transformed into a feature vector, which contains a number of features that are descriptive of the object. The number of features may not be too large, because of the curse of dimensionality; but can be large enough to accurately predict the output.
3. Determine the structure of the learned function and corresponding learning algorithm. A learning algorithm is chosen, e.g., artificial neural networks, decision trees, Bayes classifiers or support vector machines. The learning algorithm is used to build the classifier.
4. Build the classifier (e.g. classification model). The learning algorithm is run on the gathered training set. Parameters of the learning algorithm may be adjusted by optimizing performance on a subset (called a validation set) of the training set, or via cross-validation. After parameter adjustment and learning, the performance of the algorithm may be measured on a test set of naive samples that is separate from the training set.

Once the classifier (e.g. classification model) is determined as described above, it can be used to classify a sample, e.g., that of a subject described herein. In some instances, sequence data is obtained from a sample from the subject suspected having a disease or a particular stage of a disease and a classifier/classification model or algorithm (e.g., trained algorithm) is applied to the resulting data in order to detect or predict, individuals having the disease or the particular stage of the disease.

Training of multi-dimensional classifiers (e.g., algorithms) may be performed using numerous samples. For example, training of the multi-dimensional classifier may be performed using at least about 10, 20, 30, 40, 50, 60, 70, 80, 90, 100, 110, 120, 130, 140, 150, 160, 170, 180, 190, 200 or more samples. In some cases, training of the multi-dimensional classifier may be performed using at least about 200, 210, 220, 230, 240, 250, 260, 270, 280, 290, 300, 350, 400, 450, 500 or more samples. In some cases, training of the multi-dimensional classifier may be performed using at least about 525, 550, 600, 650, 700, 750, 800, 850, 900, 950, 1000, 1100, 1200, 1300, 1400, 1500, 1600, 1700, 1800, 2000 or more samples.

As trained algorithms require manipulation of many parameters simultaneously, sometimes tens or hundreds of parameters such as cfNA levels, they can be developed and calculated using appropriate software programming methods, and may be implemented on a computer. A further discussion of computer and software implements that may be used to compute or develop a trained algorithm is provided further below.

In some embodiments, the methods described herein may comprise training machine learning models. In some embodiments, the methods described herein may comprise trained machine learning models comprise a supervised machine learning model, an unsupervised machine learning model, a deep learning model, or a time-series machine learning model. The trained algorithm may comprise an unsupervised machine learning algorithm. The trained algorithm may comprise a supervised machine learning algorithm. The trained algorithm may comprise a deep learning algorithm. The trained algorithm may comprise a time-series machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise a self-supervised machine learning algorithm. The time-series machine learning algorithm may comprise autoregressive integrated moving average (ARIMA), recurrent neural networks (RNN), convolutional neural networks (CNN), Gaussian processes, long short-term memory networks, gated recurrent unit networks. Hidden Markov Models, or transformer-based models.

In some embodiments, a machine learning algorithm of a method as described herein utilizes one or more neural networks. In some case, a neural network is a type of computational system that can learn the relationships between an input dataset and a target dataset. A neural network may be a software representation of a human neural system (e.g., cognitive system), intended to capture “learning” and “generalization” abilities as used by a human. In some embodiments, the machine learning algorithm comprises a neural network comprising a CNN. Non-limiting examples of structural components of machine learning algorithms described herein include: CNNs, recurrent neural networks, dilated CNNs, fully-connected neural networks, deep generative models, and Boltzmann machines. Total number of learnable or trainable parameters;

In some embodiments, the neural network comprises artificial neural networks (ANNs). ANNs may be machine learning algorithms that may be trained to map an input dataset to an output dataset, where the ANN comprises an interconnected group of nodes organized into multiple layers of nodes. For example, the ANN architecture may comprise at least an input layer, one or more hidden layers, and an output layer. The ANN may comprise any total number of layers, and any number of hidden layers, where the hidden layers function as trainable feature extractors that allow mapping of a set of input data to an output value or set of output values. As used herein, a deep learning algorithm (such as a deep neural network (DNN)) is an ANN comprising a plurality of hidden layers, e.g., two or more hidden layers. Each layer of the neural network may comprise a number of nodes (or “neurons”). A node receives input that comes either directly from the input data or the output of nodes in previous layers, and performs a specific operation, e.g., a summation operation. A connection from an input to a node is associated with a weight (or weighting factor). The node may sum up the products of all pairs of inputs and their associated weights. The weighted sum may be offset with a bias. The output of a node or neuron may be gated using a threshold or activation function. The activation function may be a linear or non-linear function. The activation function may be, for example, a rectified linear unit (ReLU) activation function, a Leaky ReLU activation function, or other function such as a saturating hyperbolic tangent, identity, binary step, logistic, arctan, softsign, parametric rectified linear unit, exponential linear unit, softplus, bent identity, softexponential, sinusoid, sinc, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training dataset and a gradient descent or backward propagation method so that the output value(s) that the ANN computes are consistent with the examples included in the training dataset.

In some embodiments of a machine learning algorithm as described herein, a machine learning algorithm comprises a neural network such as a deep CNN. In some embodiments in which a CNN is used, the network is constructed with any number of convolutional layers, dilated layers or fully-connected layers. In some embodiments, the number of convolutional layers is between 1-10 and the dilated layers between 0-10. The total number of convolutional layers (including input and output layers) may be at least about 1, 2, 3, 4, 5, 10, 15, 20, or greater, and the total number of dilated layers may be at least about 1, 2, 3, 4, 5, 10, 15, 20, or greater. The total number of convolutional layers may be at most about 20, 15, 10, 5, 4, 3, or less, and the total number of dilated layers may be at most about 20, 15, 10, 5, 4, 3, or less. In some embodiments, the number of convolutional layers is between 1-10 and the fully-connected layers between 0-10. The total number of convolutional layers (including input and output layers) may be at least about 1, 2, 3, 4, 5, 10, 15, 20, or greater, and the total number of fully-connected layers may be at least about 1, 2, 3, 4, 5, 10, 15, 20, or greater. The total number of convolutional layers may be at most about 20, 15, 10, 5, 4, 3, 2, 1, or less, and the total number of fully-connected layers may be at most about 20, 15, 10, 5, 4, 3, 2, 1, or less.

Alternatively, an attention mechanism (e.g., a transformer) is applied to mimic human cognitive process of selectively focusing on relevant information while filtering out irrelevant details. Attention mechanisms may focus on, or “attend to,” certain input regions while ignoring others. This may increase model performance because certain input regions may be less relevant. At each operation, an attention unit can compute a dot product of a context vector and the input at the operation, among other operations. The output of the attention unit may define where the most relevant information in the input sequence is located.

In some cases, the methods described herein comprise comprising detecting a disease state or a disease stage in a subject via a trained algorithm and administering an therapy to the subject to treat the disease. In some embodiments, the therapy comprises a drug (e.g. a chemotherapeutic agent) and methods involve detecting the disease state or a disease stage in the subject via the trained algorithm and administering a new drug or a higher dose of the drug to the subject.

The present disclosure also provides for computer systems that are programmed to implement methods of the disclosure. FIG. 9 shows a computer system 901 that is programmed or otherwise configured to generate or develop antibody profile or compare antibodies with the profile of the specific immune response. The computer system 901 can regulate various aspects of the present disclosure, such as, for example, receive or generate sequence reads, correlate sequences to specific epitopes or antibodies, output a result for the user as to the presence of an antibody or profile, or an expected progression of a disease. The computer system 901 can be an electronic device of a user or a computer system that is remotely located with respect to the electronic device. The electronic device can be a mobile electronic device.

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet. The network 930 in some cases is a telecommunication and/or data network. The network 930 can include one or more computer servers, which can enable distributed computing, such as cloud computing. The network 930, in some cases with the aid of the computer system 901, can implement a peer-to-peer network, which may enable devices coupled to the computer system 901 to behave as a client or a server.

The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 401 can be included in the circuit. In some cases, the circuit is an application specific integrated circuit (ASIC).

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 901 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 901 via the network 930.

Methods as described herein can be implemented by way of machine (e.g., computer processor) executable code stored on an electronic storage location of the computer system 901, such as, for example, on the memory 910 or electronic storage unit 915. The machine executable or machine-readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” generally in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 935 that comprises a user interface (UI) 940 for providing, for example, selecting antibodies for analysis, interacting with graphs correlating antibodies to specific generated profiles. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 905.

### EXAMPLES

### Example 1—Detection of Disease Status in a Subject

A subject having or suspected of having a disease is provided. A sample of DNA fragments (e.g. a sample of cfDNA sequences obtained from a blood or blood-derived fluid, or a sample of genomic DNA fragments provided from a tissue biopsy) is obtained from the subject. A sequencing method (e.g. any of the sequencing methods described herein) specifically identifying: (i) 5-hydroxymethylcytosine (5hmC); or (ii) 5hmC and 5-methylcytosine (5mC) bases in said DNA is executed on the DNA fragment sample to provide locations or abundances of 5hmC or 5hmC/5mC in DNA fragments of the DNA fragment sample. A trained classifier is applied to the locations or abundance information for 5hmC or 5hmC/5mC; the trained classifier has previously been trained on location or abundance information 5hmC or 5hmC/5mC in regions across a genome of a patient associated with disease status (e.g. identified by comparing methylation levels in a patient with the disease compared to a subject without the disease), regions associated with disease stage (e.g. identified by comparing methylation levels in a patient with an early stage of the disease vs a patient with a late stage of the disease), or regions associated with minimum residual disease (e.g. identified by comparing methylation levels in a patient with the disease pre-treatment vs a patient with the disease post-treatment). An example of disease stage can include, but it not limited to, a neurological disease (e.g. relapsing-remitting multiple sclerosis versus secondary progressive multiple sclerosis) or a cancer (e.g. stage I, II, III, or IV, or an early-stage cancer versus a late-stage cancer).

### Example 2.—5-Methylcytosine and 5-Hydroxymethylcytosine as Synergistic Biomarkers for Early Detection of Colorectal Cancer

### Abstract

Early cancer detection has the potential to significantly improve treatment outcomes and survival rates. This study investigates the roles of 5-methylcytosine (5mC) and 5-hydroxymethylcytosine (5hmC) as biomarkers for early-stage colorectal cancer (CRC) detection in cell-free DNA (cfDNA). Using whole genome sequencing, analyzed cfDNA from 37 treatment-naive CRC patients and 32 healthy controls were analyzed. The findings indicate that combining measurements of 5mC and 5hmC significantly enhances diagnostic accuracy (AUC=0.95) compared to other approaches that conflate these markers (e.g. approaches which merely denote 5mC as 5hmc as “modC” without distinguishing between the two, AUC=0.66). Notably, 71.7% of differentially methylated regions (DMRs) exhibiting an increase in 5hmC in stage I cfDNA showed a corresponding decrease in 5mC in stage IV, suggesting that 5hmC can effectively track regions undergoing demethylation during tumor development. These results support the hypothesis that distinguishing between 5mC and 5hmC can improve the sensitivity of tests for early cancer detection (e.g. liquid biopsy tests).

### Introduction

Early detection has the potential to transform the treatment and survival of cancer. Cancer is caused by changes to the genome and epigenome, characterized by somatic mutations. Cancer-associated somatic mutations affect epigenetic systems in hematological and solid tumors, and epigenetic modifications are targeted by epi-drugs in cancer therapy. Recent evidence also suggests that epigenetic perturbation can drive cancer initiation independently of somatic mutations.

DNA cytosine methylation and demethylation result in chemically stable epigenetic changes that can be accurately measured. These modifications predominantly occur at CpG dinucleotides, where cytosines are followed by guanines in the DNA sequence. Cytosine methylation results in 5-methylcytosine (5mC) and enzymatic oxidation converts 5mC to 5-hydroxymethylcytosine (5hmC), which upon further oxidation generates cytosine derivatives that are removed by repair pathways resulting in overall demethylation (FIG. 1 panel a). 5mC and 5hmC play distinct roles in epigenetic regulation, although this information is often lost as they are typically (mis)read as a combined signal in classical sequencing approaches. 5mC is typically found at gene promoters where it is associated with gene repression. In contrast, 5hmC is enriched at poised or active enhancers and gene bodies of actively transcribed genes and plays a critical role in the transcriptional reprogramming of cells that are undergoing state transitions.

Methylation is used as a biomarker for early cancer detection. Epigenetically reprogrammed tumor cells release DNA into the bloodstream, where methylation changes can be detected in cell-free DNA (cfDNA). Despite progress, early-stage cancers remain difficult to detect. For example, DNA methylation-based liquid biopsy tests for colorectal cancer (CRC) exhibit 99.5% specificity, showing 43.3% sensitivity at stage I compared to 95.3% sensitivity at stage IV (Table 2). The observed lower sensitivity for early-stage cancer, compared to later stages, can be due to the limited amount of tumor-derived cfDNA, subtler epigenetic shifts at this stage as measured by modC (where modC is the combined signal from 5mC+5hmC), or a combination of both factors. This issue is compounded by the limitations of current methods used to assay methylation. For example, enrichment approaches typically resolve DNA fragments, rather than individual bases, and suffer from limited accuracy in identifying and measuring differentially methylated regions (DMRs). In addition, bisulfite sequencing conflates 5-methylcytosine (5mC) and 5-hydroxymethylcytosine (5hmC) into a single modC signal (e.g. modC=5mC+5hmC), masking the individual contributions of these two modifications particularly in early-stage disease (FIG. 1 panels b and c).

TABLE 2
Sensitivity and specificity for CRC stage I detection as analyzed in Example 2. N is the
number of samples assessed. ND is not disclosed. Grail refers to the Circulating Cell-free
Genome Atlas study (NCT02889978; Klein et al, 2021), Freenome to the PREEMPT study
(NCT04369053), and Guardant to the ECLIPSE study (Chung et al, 2024).

| | Stage I | | | Stage IV | | |
|---|---|---|---|---|---|---|
| Reference | Specificity | Sensitivity | N | Specificity | Sensitivity | N |
| Grail | 99.5% | 43.3% | 30 | 99.5% | 95.3% | 64 |
| Freenome | 91.5% | 57.1% | ND | ND | 100% | ND |
| Guardant | 89.9% | 55% | 22 | 89.9% | 100% | 10 |

Given the different biological roles of 5mC and 5hmC, it was hypothesized that discriminating these changes at base resolution would provide more informative data and increase the sensitivity of early-cancer detection. To test this hypothesis, performed whole genome sequencing (WGS) of liquid biopsy samples from stage I CRC cancer patients and healthy controls was performed using a method that simultaneously sequences 5mC and 5hmC at read level. The results demonstrate that 5hmC provides informational value that is additive to 5mC, with models utilizing both 5mC and 5hmC achieving higher diagnostic accuracy than modC (AUC 0.95 versus 0.66).

### Results

To test the hypothesis that separate measurement of 5mC and 5hmC would provide more useable information to detect the presence of early-stage cancer in plasma, differences in 5mC and 5hmC in cfDNA from individuals with colorectal cancer and healthy controls were examined. For this purpose, cfDNA from 69 samples including 32 healthy controls and 37 treatment naive CRC patients (26 stage I and 11 stage IV) was analyzed. Demographics of the cohort including age, sex, and relevant clinical characteristics such as smoking history, are outlined in Table 1. For each sample. 10 ng of cfDNA was processed using the duet evoC assay (biomodal), and libraries were subjected to WGS to a mean depth of 34×.

TABLE 1
Demographics and covariates in the cohort analyzed in Example 2.

| | | | | | CRC | | CRC | |
|---|---|---|---|---|---|---|---|---|
| | Total | | Control | | stage I | | stage IV | |
| N | 69 | | 32 | | 26 | | 11 | |
| Age (years), | 60.8 | (6.6) | 58.8 | (6.9) | 61.5 | (6.0) | 65.5 | (4.8) |
| mean (SD) | | | | | | | | |
| Female gender, | 48 | (83) | 25 | (78) | 23 | (88) | 9 | (82) |
| n (%) | | | | | | | | |
| Ethnicity, | | | | | | | | |
| n (%) | | | | | | | | |
| White | 69 | (100) | 32 | (100) | 26 | (100) | 11 | (100) |
| Alcohol | | | | | | | | |
| consumption, | | | | | | | | |
| n (%) | | | | | | | | |
| Unknown | 6 | (9) | 5 | (16) | 1 | (4) | 0 | (0) |
| None | 17 | (25) | 3 | (9) | 8 | (31) | 6 | (55) |
| Light ≤ | 46 | (67) | 24 | (75) | 17 | (65) | 5 | (46) |
| drink/day | | | | | | | | |
| Smoking, | | | | | | | | |
| n (%) | | | | | | | | |
| Never smoked | 57 | (83) | 24 | (75) | 23 | (89) | 10 | (91) |
| Ex-smoker | 6 | (9) | 4 | (13) | 2 | (8) | 0 | (0) |
| Smoker | 6 | (9) | 4 | (13) | 1 | (4) | 1 | (9) |

To focus the analysis on regions where significant epigenetic shifts occur, regions were first identified which showed significant differences in modC using publicly available data from 9 stage IV CRC samples with matched normal tissue analysed by Illumina 450K arrays. Overall, the goal was to track regions undergoing methylation changes, which can be associated with tumor progression. Overall, 11,691 modC DMRs (q<0.05) were found, of which 7,961 were hypo-methylated and 3,743 were hyper-methylated in CRC (FIG. 2 panel a). Each of these regions contained one or more CpGs and spanned tens to thousands of base pairs (bp), with a mean size of 857 bp (FIG. 4)

Differences in 5mC, 5hmC, and modC between plasma from CRC patients and healthy controls were then analyzed across the 11,691 modC DMRs identified in public tissue data, performing separate comparisons for stage I and stage IV. A strong correlation was found between 5mC and modC DMRs in stage IV plasma and the stage IV modC tissue data (both R²=0.66). However, no correlation was observed when comparing 5mC or modC DMRs in stage I plasma with stage IV tissue (both R²<0.001) (FIG. 5). This difference can be explained by methylation changes during tumor progression, in addition to the higher proportion of tumor DNA in the cfDNA of individuals with late-stage cancer (Diehl et al 2008, Bettegowda et al 2014).

After confirming that 5mC DMRs in stage IV tissue were similarly altered in stage IV plasma, 5mC and 5hmC DMRs in stage I plasma were analyzed to assess their relationship to the changes observed in stage IV plasma. This was done to determine whether patterns of changes in 5mC or 5hmC at stage I preceded the methylation changes seen in late-stage cancer, potentially acting as an early indicator of ctDNA presence in plasma. Of the 10,557 modC tissue DMRs that showed statistically significant differences in 5mC in stage IV plasma (q<0.05), it was found that 41.2% (4,346/10,557) also had a significant difference in 5mC, and 25% (2,643/10,557) also had a significant difference in 5hmC in stage I plasma. While there was some overlap between the sets of stage I 5mC and 5hmC DMRs, 48% (1,268/2,643) of the 5hmC DMRs did not show statistically significant differences in 5mC. This suggests that measuring 5hmC in stage I plasma provides complementary information to that obtained from 5mC (FIG. 2 panel b).

The direction of DMRs (e.g. increase or decrease in methylation) between stage I and IV plasma were then compared. Among the 4,436 5mC DMRs in stage I plasma, 44.6% (1,938/4,436) were consistently methylated or demethylated in both stage I and stage IV plasma. Among the regions which did not show the same direction of change in 5mC in stage I and stage IV, a majority (43.9%; 1,908/4,436) showed an increase in 5mC in stage I plasma, which subsequently decreased in 5mC in stage IV plasma (FIG. 2 panel c).

Given 5hmC is an intermediate of de-methylation, it follows that changes in 5hmC at stage I can precede loss of 5mC at stage IV. Supporting this, the majority (71.7%; 1.894/2,643) of DMRs with statistically significant changes in 5hmC in stage I and 5mC in stage IV plasma had an increase in 5hmC at stage I and a decrease in 5mC at stage IV. Of the remaining 5hmC DMRs, 26% (687/2,643) showed an increase in 5hmC in stage I and an increase in 5mC in stage IV plasma. A small proportion of the regions (2.3%; 62/2,643) showed a statistically significant decrease in 5hmC in stage I plasma. Collectively, these data are consistent with 5hmC being an intermediate in the transition from methylated to unmethylated C during the progression of disease from stage I to stage IV. In particular, a discernible increase in 5hmC in stage I plasma in these regions appears to be a clear marker of regions which become demethylated in late-stage colorectal cancer (FIG. 2 panels c and d). Taken together, the analysis of DMRs suggests that 5mC and 5hmC each provide complementary information that can be used to identify, dynamic methylation changes in cfDNA.

Motivated by this, whether a combination of features based on 5mC and 5hmC provided greater discriminatory power to identify stage I CRC patients from healthy controls was then assessed. Generalized linear models were built using the 11,691 TCGA-derived DMRs and applied a leave-one-out cross-validation approach (LOOCV) to assess model performance on stage I cfDNA samples. In LOOCV, the model is iteratively trained on all samples except one, and the left-out sample is used for testing. This process is then repeated for each sample in turn. To address variability in the learning (e.g. trained) algorithm, predictions were averaged across multiple random seeds to generate the prediction for the held-out test sample. The classifier demonstrated an AUC of 0.54 using 5hmC alone, 0.70 with 5mC alone, 0.66 with modC, and 0.95 using each of 5mC and 5hmC (e.g., independent features from both 5mC and 5hmC) (FIG. 3 panel a). CMS guidelines for blood-based biomarker testing for CRC detection mandate a minimum 74% sensitivity and 90% specificity. At 95% specificity, the 5mC and 5hmC model had a sensitivity of 85% (FIG. 3 panel b). In comparison, other publicly disclosed liquid biopsy tests reflect ongoing challenges in robust early-stage detection.

To assess each model's sensitivity to sample selection 500 sub-cohorts were further generated, each with 52 samples, by randomly excluding three CRC samples and three healthy controls per sub-cohort. Each sub-cohort was modelled across multiple random seeds and a mean AUC calculated. Although variance in AUC across the different cohorts was observed, models that used each of 5mC and 5hmC had consistently higher AUCs (FIG. 3 panel c).

Similar models were next built to distinguish stage IV CRC patients from healthy controls, as well as stage I from stage IV patients (FIG. 6). In the comparison of stage IV patients with healthy controls, the highest AUC was achieved with the model using modC (AUC=0.88), though the contribution of 5mC alone was nearly equivalent (AUC=0.83). This suggests that 5mC provides the majority of the discriminatory signal at later stages of disease. In contrast, 5hmC contributed significantly to distinguishing between stage I and stage IV CRC, with similar AUCs for 5hmC alone (AUC=0.95), and the combination of 5hmC and 5mC (AUC=0.91). These results support the hypothesis that 5hmC offers additional discriminatory power in the earlier stages of cancer progression, consistent with its value in differentiating stage I patients from healthy controls. Taken together, these data suggest that models including 5mC and 5hmC as distinct biomarkers improve the performance of early-stage CRC detection.

### Discussion

In this study, a goal was to evaluate whether base resolution information on 5mC and 5hmC provides additional diagnostic value, relative to 5mC alone, 5hmC alone, or modC, for cancer (eg. stage 1 CRC) detection using cfDNA. Multiple regions that transitioned from being methylated or hydroxymethylated in stage I to demethylated in stage IV CRC cfDNA were observed. Since 5hmC can be a transitional intermediate on the demethylation pathway from 5mC to C, it was hypothesized that 5mC and 5hmC can provide independent and additive features that can be used to improve classifier performance for early detection. Models using both 5mC and 5hmC features outperformed those based on modC, 5mC, or 5hmC alone. Notably, models used to detect stage I CRC from healthy controls frequently selected 5hmC features, highlighting the distinct contribution of 5hmC to improved classification performance (FIG. 6).

This study leveraged the duet evoC assay to distinguish between 5mC and 5hmC, allowing for the analysis of NGS data using modC, 5mC, 5hmC, and both 5mC and 5hmC models. This flexible approach provides a powerful tool for discovery, where different indications may exhibit different levels of signal from 5mC, 5hmC, and genetics. Building on the discovery operation, it is then possible to scale an assay that focuses specifically on the combination of methylation and genetics that yield the required signal.

In conclusion, distinguishing between 5mC and 5hmC enhanced sensitivity for early-stage cancer (e.g. CRC) detection, showing improved performance compared to modC models. The data supports the use of sequencing methods that distinguish 5mC and 5hmC in early detection.

### Methods

### Cohort

This is a single center case-control retrospective cohort using cfDNA extracted from double spun plasma from consented patients undergoing routine screening for CRC by colonoscopy. Samples were obtained from National BioService LLC. Control samples were from individuals aged 45-85 years who were at average risk for CRC and had been assessed by colonoscopy, with results that showed no presence of CRC or adenomatous polyps. Cancer samples were obtained from individuals aged 45-85 years who underwent colonoscopy and were diagnosed with CRC. The following data were available for each blood sample donor: age, sex, ethnicity, and smoking status. The study was performed in accordance with the Declaration of Helsinki and was approved by the relevant independent ethics committee. Written informed consent was obtained from all donors of samples. 69 samples were selected balanced for key characteristics (age, sex and diagnosis).

### Sample Preparation

Where possible, blood was sampled before colonoscopy. 10 mL blood was drawn into a K2 EDTA blood tube, placed on ice, and processed within 4 hours. Samples were centrifuged (2,000 g for 10 min at room temperature). The plasma layer was transferred to a clean tube and was again centrifuged (2,000 g for 10 min at room temperature) to remove any remaining cellular material. Double-spun plasma was aliquoted into tubes in volumes of at least 1 mL and then immediately frozen and stored at −80° C. cfDNA was extracted using the Chemagic™ cfDNA 5k Kit (Revvity) using between 1 and 7 mL of plasma. Resulting cfDNA was quantified using a Qubit Fluorometer (Life Technologies) and quality was assessed using Agilent TapeStation and Cell-free DNA Screen Tape assay, with samples included if they had >70% for the % cfDNA quality metric on the Cell-free DNA ScreenTape assay on the Agilent TapeStation.

### Library Preparation and Sequencing

The duet evoC assay was run according to manufacturer's instructions (see Fullgrabe et al., 2023). In brief, 10 ng of each cfDNA sample was end-repaired. A-tailed, and ligated to hairpin adapters. Adaptors were digested and the 3′ hydroxyl group was extended by a DNA polymerase to generate molecules with the original sample DNA strand connected to its (copied) complementary strand. Y-shaped sequencing adapters were then ligated to molecules. Methylation at 5mC was enzymatically copied across the CpG unit to the C on the copy strand using a DNA methyltransferase, whereas 5hmC was glycosylated (e.g. enzymatically glycosylated) via beta-glycosyltransferase to prevent such a copy. 5mC on both the original and copy strands was then also protected (e.g. glycosylated) from subsequent deamination. Unmodified Cs were then deaminated to uracil, subsequently read as thymine via the joint action of TET2, APOBEC3A, and UvrD helicase.

After PCR, libraries were analyzed by Qubit and TapeStation and pooled at equimolar concentration. High throughput sequencing was performed on a NovaSeq 6000 using an S4 flow-cell with 2×150 reads, with 8 libraries per flow-cell, yielding an average of 1.3 billion read pairs (+0.24 billion) per library or a mean genome coverage of 34× (SD 7.3×). Sensitivity and specificity of 5mC conversion were assessed by spike-in controls and were within expected ranges: 5mC sensitivity 96.7%±0.8%; 5hmC sensitivity 98.15%±0.5%; modC specificity 99.6%±0.15%. 5mC sensitivity was calculated by counting the proportion of 5mCpGs identified on a fully methylated lambda genome, 5hmC sensitivity was calculated by counting the proportion of 5hmCpGs identified on a synthetic oligonucleotide, and specificity was calculated by counting the proportion of unmodified CpGs identified on an unmethylated pUC19 genome.

Fastq files were processed using biomodal's duet pipeline (version 1.3.0), as described in (Fillgrabe et al., 2023.

### Cohort Validation

For each individual in the cohort, the agreement between the reported sex was assessed by comparing the median coverage of chromosomes X and Y. Additionally, for healthy controls and stage I CRC plasma samples, ctDNA estimates were obtained using ichorCNA (Adalsteinsson et al., 2017). All samples had estimated tumor fractions below the ichorCNA detection threshold set at a maximum of 2.037% for healthy controls and 4.6% for stage 1 CRC patients. This threshold was not applied for stage IV CRC plasma samples.

### Feature Generation

DNA methylation data of paired tumor and normal samples of patients with stage IV CRC was downloaded from TCGA (https://portal.gdc.cancer.gov/, January 2024). These included methylation data of 9 paired tumor-normal samples using Illumina Infinium Human Methylation 450K BeadChip (Illumina 450K array). Methylation probes without any beta values in any sample were filtered out. DMRs between stage IV and normal samples were identified using DMRcate (Peters et al, 2021). Significant DMRs with FDR<0.05 were lifted over from hg19 to GRCh38 genomic coordinates. Epigenetic data (modC, 5mC, and 5hmC fractions) was summarized across regions identified as significantly differentially methylated in paired stage IV CRC tissue samples. Average modC, 5mC, and 5hmC fractions for each region were obtained by dividing the sum of the counts of each cytosine modification by the count of all cytosines across all CpGs in the reads covering each region of interest.

### Model Building

Model building was carried out using the R package Glmnet (Friedman et al., 2010; Tay et al, 2023). Briefly, given the high dimensionality of the dataset (~11 k features×58 samples) a regularization operation was performed to decrease the number of features used to build each model. Feature selection was carried out using a lasso logistic regression model. The regularisation strength lambda was chosen by cross-validation on the training set, with number of folds set to 10. The best lambda value was chosen as lambda.min, unless no features were retained, in which case lambda was chosen as the minimum lambda returning a model with at least 1 feature.

For evaluation of the different feature sets, a leave-one-out (LOOCV) approach was used. In this method, the model is trained on all but one sample in the dataset and tested on the remaining sample. This process is repeated for each sample in turn, meaning that each sample serves as a test set once. To address variability in the training process, predictions were averaged across 25 random seeds. The predicted probabilities (corresponding to the probability a sample was stage 1 CRC according to the model) for each sample were used to generate the ROC curves and AUC scores. To address the reproducibility of the results to different starting cohorts, 500 different sub-cohorts were generated by removing a random subset of 6 samples (3 with stage I CRC and 3 healthy controls) from the main cohort and evaluating each feature set.

### DMR Calling

Differential modification (i.e. 5mC, 5hmC, or modC) calling in cfDNA data was carried out by aggregating counts of modified and unmodified cytosines across all CpG contexts within the regions defined by the analysis of stage IV TCGA samples.

A logistic regression model was employed to analyze the aggregated data. The model is structured as follows assuming N samples separated into two groups. For each sample i, the proportion of modified bases in the region pᵢ is modelled via the logistic regression model:

$$log ( pi / ( 1 - pi ) ) = β 0 + β GXG , i …$$

In this equation, β₀ is the intercept and β_{G}X_{G,i} represents the group term (e.g., stage IV vs. control, where X_{G,i} would be 0 if sample i belongs to the control group, and 1 if it belongs to the stage IV group).

For each base or region of interest, an independent statistical test was performed to evaluate the null hypothesis H₀: β₁=0. If the null hypothesis was rejected, it indicates that the log-odds (and consequently, the methylation proportions) differ significantly between the treatment and control groups. In this case, the region is classified as a differentially 5mC or 5hmC modified region (DMR). Conversely, if the null hypothesis is not rejected, it suggests that there is no statistically significant difference in methylation levels between the two groups for that particular region. DMR calls were corrected for multiple testing using Benjamini-Hochberg, resulting q-values were called based on a predetermined p-value threshold. DMRs were called for both 5mC and 5hmC separately.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of processing a nucleic acid sample from a subject, comprising:
(a) obtaining the nucleic acid sample from the subject, wherein the nucleic acid sample comprises cell-free DNA (cfDNA) sequences; and
(b) sequencing the nucleic acid sample to generate a plurality of sequence reads.

2. The method of claim 1, wherein the nucleic acid sample is obtained from a plasma, serum, or whole blood sample from said subject.

3. The method of claim 1, wherein the subject is suspected of having cancer.

4. The method of claim 3, wherein the cancer is selected from the group consisting of colorectal cancer, gastric cancer, lung cancer, liver cancer, ovarian cancer, breast cancer, prostate cancer, esophageal cancer, thyroid cancer, uterine cancer, pancreatic cancer, bladder cancer, hematological cancer, brain cancer, and kidney cancer.

5. The method of claim 5, further comprising attaching a hairpin to strands of said cfDNA fragments such that said strands are covalently linked from 5′ to 3′, thereby producing hairpin-linked cfDNA sequences.

6. The method of claim 6, further comprising digesting said hairpin-linked cfDNA fragments to generate hairpin-linked strands of said cfDNA sequences.

7. The method of claim 7, further comprising extending said harpin-linked strands of said cfDNA fragments to produce double-stranded hairpin-linked cfDNA fragments.

8. The method of claim 8, further comprising adding a non-deaminatable moiety to 5hmC of said double-stranded hairpin-linked cfDNA sequences.

9. The method of claim 9, wherein said adding said non-deaminatable moiety to said 5hmC of said double-stranded hairpin-linked cfDNA fragments comprises adding a sugar residue, a glucose molecule, or a glucose-derivative donor substrate to said 5hmC of said double-stranded hairpin-linked cfDNA sequences.

10. The method of claim 10, further comprising contacting said double-stranded hairpin-linked cfDNA fragments with a DNA methyltransferase.

11. The method of claim 11, further comprising contacting said double-stranded hairpin-linked cfDNA sequences with a dioxygenase and a glycosyltransferase.

12. The method of claim 12, wherein said dioxygenase comprises a TET enzyme.

13. The method of claim 1, wherein (b) comprises performing a next generation sequencing reaction.

14. A composition comprising:
(a) a nucleic acid;
(b) a methyltransferase;
(c) a dioxygenase;
(d) an alkylating agent; and
(e) a glycosyltransferase.

15. The composition of claim 1, wherein the alkylating agent is selected from the group consisting of busulfan, improsulfan, piposulfan, benzodizepa, carboquone, meturedepa, uredepa, altretamine, triethylenemelamine, triethylenephosphoramide, triethylenethiophosphoramide, trimethylolmelamine, chlorambucil, chlomaphazine, cyclophosphamide, estramustine, iphosphamide, mechlorethamine, mechlorethamine oxide hydrochloride, melphalan, novembichine, phenesterine, prednimustine, trofosfamide, carmustine, chlorozotocin, fotemustine, lomustine, nimustine, and ranimustine.
